(19) United States
(12) Patent Application Publication
(10) Pub. No.: US 2010/0254929 A1
(43) Pub. Date: Oct. 7, 2010

(54) METHOD FOR MAKING UP THE EYELASHES COMPRISING THE APPLICATION OF 2 COMPOSITIONS

(75) Inventor: Olivier Raineau, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'Oreal, Paris (FR)

(21) Appl. No.: 12/742,553

(22) PCT Filed: Nov. 12, 2008
(86) PCT No.: PCT/EP2008/065362

§ 371 Date: May 12, 2010

Related U.S. Application Data
(60) Provisional application No. 61/014,492, filed on Dec. 18, 2007.

(30) Foreign Application Priority Data
Nov. 13, 2007 (FR) 0759013

(51) Int. Cl. A61K 8/92; A61Q 1/10; A45D 40/26

(52) U.S. Cl. 424/70.7; 132/216

(57) ABSTRACT

A subject-matter of the present invention is a cosmetic method for making up or for the nontherapeutic care of keratinous fibres comprising the application, to the keratinous fibres: of a first composition comprising a cosmetically acceptable medium and of a second composition comprising at least one compound or a mixture of compounds which, when the composition is brought to a temperature of greater than or equal to 40° C., confers, on the said composition, a dmax threading nature of greater than or equal to 5 mm, the said second composition being, prior to, simultaneously with or subsequent to the application thereof, brought to a temperature of greater than or equal to 40° C.

A subject-matter of the present invention is a cosmetic method for making up or for the nontherapeutic care of keratinous fibres (such as the eyelashes, eyebrows or hair) comprising the application, to keratinous fibres, of a first cosmetic composition and of a second cosmetic composition. Another subject-matter of the present invention is a method for removing make-up films formed by the application of the said compositions.

The compositions employed in the method according to the invention are provided in particular in the form of a product for the eyelashes'or mascara.

The term “mascara” is understood to mean a composition intended to be applied to keratinous fibres: it can be a composition for making up keratinous fibres, a base for making up keratinous fibres or base coat, a composition to be applied to a mascara, also referred to as top coat, or alternatively a composition for the cosmetic treatment of keratinous fibres. The mascara is more particularly intended for human keratinous fibres but also for false eyelashes.

The prior art describes mascara compositions comprising fibres in order to obtain an elongating effect on the eyelashes. These fibres can add a small amount of physical length to the eyelashes when they are sufficiently rigid and visible and when they occur at the end of the eyelash. However, the gain in physical elongation obtained via such mascaras remains moderate as it is difficult to orientate the fibres in order to stack them at the end of the eyelash. Furthermore, the presence of fibres can reduce the adhesion of the mascara to the eyelashes, lengthening the time necessary for making up.

Another technical route described in the document EP 1 430 868 is the use of mascaras exhibiting a “threading” nature at ambient temperature and which are capable of forming, when applied to keratinous fibres and after drawing out using a brush, threads in the extension of the eyelashes, without use of a heat source.

However, the ability of these mascaras to form threads at ambient temperature does not simplify their use: in particular, when it is withdrawn in order to be applied, the mascara can form threads between the container in which it is present and the applicator or between the eyelashes and the applicator.

Moreover, control of the length of the threads formed on the eyelashes is problematic as the threads do not spontaneously break. In addition, the latter rarely exhibit a stiffness sufficient to remain aligned in the extension of the eyelash and to make possible a lasting elongating effect.

The inventors have discovered that the properties described above can be obtained by using a composition exhibiting a specific threading nature under the action of a heat source. This composition, after application to the eyelashes and in combination with a heat source, makes it possible to obtain threads in the extension of the eyelashes, so as to create an elongation of the eyelash.

However, the films formed by these compositions are more difficult to remove than conventional products.

The aim of the present invention is to provide a method for coating keratinous fibres which makes it possible to obtain a deposited layer on the eyelashes which exhibits a good elongating effect on the eyelashes, in particular under the effect of heat, and good hold over time, the said deposited layer being easy to remove.

The inventors have discovered that the properties described above can be obtained by using a first composition comprising a cosmetically acceptable medium and a second composition exhibiting a specific threading nature under the action of a heat source.

More specifically, a subject-matter of the invention is a cosmetic method for making up or for the nontherapeutic care of keratinous fibres comprising the application, to the keratinous fibres:
of a first composition comprising a cosmetically acceptable medium and
of a second composition comprising at least one compound or a mixture of compounds which, when the composition is brought to a temperature of greater than or equal to 40° C., confers, on the said composition, a dmax threading nature of greater than or equal to 5 mm, the said second composition being, prior to, simultaneously with or subsequent to the application thereof, brought to a temperature of greater than or equal to 40° C.

The threading nature represents the ability of the composition, once subjected to a heat source, to form, on the keratinous fibres, threads which, after drawing out using an applicator, are sufficiently consistent and retain their form. The use of heat so as to bring the composition to a temperature greater than or equal to 40° C. makes it possible to control the length of the threads formed in the extension of the eyelashes.

In particular, after application of the softened composition and drawing out the threads, the latter solidifiy at ambient temperature in the extension of each eyelash and make it possible to obtain a noteworthy elongating effect.

The application, prior to the second composition, of a first composition comprising hard heat-resistant particles makes it possible to form a first film on the eyelashes which withstands the heat when the second composition is subjected to a heat source. A first film (base film) which is less cohesive than the second film formed by the second composition above the base film is obtained on the eyelashes, which facilitates the removal of the combined deposited layer on the eyelashes.

The second composition can in particular be used in combination with a heating instrument, such as a heating brush, which can be applied to the eyelashes before, during or after the coating of the latter with the composition, or packaged in a device which makes it possible to apply the composition under warm conditions.

The second composition can be applied over the whole or over the upper end of the keratinous fibres, in particular of the eyelashes. According to one embodiment, the second composition is applied over the upper end of the eyelashes. The first composition is preferably applied over the whole of the eyelash.

The first and second compositions comprise a physiologically acceptable medium, that is to say a medium which is nontoxic and capable of being applied to keratinous fibres, such as the eyelashes, eyebrows and hair of human beings, in particular a medium compatible with the ocular region.

The first composition can be coloured (that is to say, comprising at least one colouring material as defined below) or uncoloured.

According to one embodiment, the second cosmetic composition is coloured, so as to form coloured threads at the end of the eyelashes.

According to another embodiment, the method according to the invention consists in:
applying an uncoloured cosmetic composition to the keratinous fibres, the said composition comprising at least one compound or a mixture of compounds which, when the composition is brought to a temperature of greater than or equal to 40° C., confers, on the said composition, a dmax threading nature of greater than or equal to 5 mm,
bringing the said composition, prior to, simultaneously with or subsequent to the application thereof, to a temperature of greater than or equal to 40° C., so as to form colourless threads at the end of the eyelashes,
then, after cooling the threads, making up the keratinous fibres and the colourless threads set in their extension with a third composition comprising one or more colouring materials, so as to colour the threads.

In particular, the second composition comprising at least one compound or a mixture of compounds which, when the composition is brought to a temperature of greater than or equal to 40° C., confers, on the said composition, a dmax threading nature of greater than or equal to 5 mm, is applied over the upper end of the eyelashes.

It is thus possible, according to this embodiment, to make-up the uncoloured threads formed over the eyelashes or at the end of the eyelashes (extension of eyelashes) with a deposited layer of a third coloured composition and then to remove the said deposited layer of third composition alone, without removing the combination of the eyelashes and extensions, so as to be able to subsequently make-up again the threads formed at the end of the eyelashes, for example with another composition which is different in colour.

Another subject-matter of the present invention is a kit for making up and/or caring for keratinous fibres, in particular the eyelashes or eyebrows, comprising:
a first composition comprising a cosmetically acceptable medium,
a second composition for making up and/or caring for keratinous fibres, the said composition comprising at least one compound or a mixture of compounds which, when the composition is brought to a temperature of greater than or equal to 40° C., confers, on the said composition, a dmax threading nature of greater than or equal to 5 mm, and
a device for the application of the said make-up and/or care compositions; and/or heating means for bringing, prior to, simultaneously with or subsequent to the application thereof, the said second composition to a temperature of greater than or equal to 40° C.

The second composition can be brought to a temperature of greater than or equal to 40° C. prior to, simultaneously with or subsequent to the application thereof to the first layer of first composition, in particular using an applicational device comprising heating means, such as a heating brush.

I) First Composition

According to one embodiment, the first composition comprises at least one compound, referred to as “first compound”, chosen from a) oils, b) compounds exhibiting a first-order phase transition temperature, in particular a melting point, and/or a glass transition temperature greater by at least 3° C., preferably by at least 5° C., than the temperature to which the second composition is brought so as to form colourless threads at the end of the eyelashes, and their mixtures.

Thus, this temperature can, for example, be greater than or equal to 45° C., better still greater than or equal to 50° C. and even better still greater than or equal to 60° C.

The first compound(s) can be present in a content ranging from 0.1 to 70% by weight, with respect to the total weight of the first composition, preferably from 0.5 to 60% by weight and better still from 1 to 50% by weight.

Preferably, the first compound is present in a content of greater than or equal to 10% by weight, with respect to the total weight of the first composition, preferably greater than or equal to 15% by weight, better still 20% by weight and even better still 25% by weight.

a) Oils

The term “oil” is understood to mean a nonaqueous fatty substance which is liquid at ambient temperature (25° C.) and atmospheric pressure (760 mmHg).

The oil can be chosen from volatile oils and/or nonvolatile oils, and their mixtures. It can be chosen from hydrocarbon oils, silicone oils, fluorinated oils or their mixtures, as described later in the description.

The oil or oils can be present in the first composition in a content ranging from 0.1 to 70% by weight, preferably from 1 to 40% by weight and better still from 5 to 20% by weight, with respect to the total weight of the first composition.

Preferably, the oil is nonvolatile.

According to one embodiment, the oil is chosen from ester oils, in particular from esters of monocarboxylic acids with monoalcohols and polyalcohols.

Advantageously, the said ester corresponds to the following formula (IV):

R₁—CO—O—R₂ (IV)
where R₁ represents a linear or branched alkyl radical of 1 to 40 carbon atoms, preferably of 7 to 19 carbon atoms, which optionally comprises one or more ethylenic double bonds and which is optionally substituted, and
R₂ represents a linear or branched alkyl radical of 1 to 40 carbon atoms, preferably of 3 to 30 carbon atoms and better still of 3 to 20 carbon atoms which optionally comprises one or more ethylenic double bonds and which is optionally substituted.

The term “optionally substituted” is understood to mean R₁ and/or R₂ can carry one or more substituents chosen, for example, from groups comprising one or more heteroaotoms chosen from O, N and S, such as amino, amine, alkoxy or hydroxyl.

Preferably, the total number of carbon atoms of R₁+R₂ is ≧9.

R₁ can represent the residue of a fatty acid, preferably a higher fatty acid, which is linear or preferably branched and which comprises from 1 to 40 and better still from 3 to 19 carbon atoms and R₂ can represent a linear or preferably branched hydrocarbon chain comprising from 1 to 40 carbon atoms, preferably from 3 to 30 carbon atoms and better still from 3 to 20 carbon atoms. Again, preferably, the number of carbon atoms of R₁+R₂≧9.

Examples of the R₁ groups are those derived from fatty acids chosen from the group consisting of acetic, propionic, butyric, caproic, caprylic, pelargonic, capric, undecanoic, lauric, myristic, palmitic, stearic, isostearic, arachidic, behenic, oleic, linolenic, linoleic, eleostearic, arachidonic, erucic and neopentanoic acids and their mixtures.

Examples of esters are, for example, purcellin oil (cetearyl octanoate), isononyl isononanoate, isopropyl myristate, 2-ethylhexyl palmitate, 2-octyldodecyl stearate, 2-octyldodecyl erucate, isostearyl isostearate and heptanoates, octanoates, decanoates or ricinoleates of alcohols or of polyalcohols, for example of fatty alcohols.

In particular, the oil is octyldodecyl neopentanoate.
b) Compounds Exhibiting a First-Order Phase Transition Temperature Greater by at Least 3° C. Than the Temperature to which the Second Composition is Brought

The first-order phase transition temperature of the first compound can range up to 2000° C.

Preferably, the first compound is provided in the form of particles which are solid at 25° C.

The compounds exhibiting a first-order transition temperature, in particular a melting point, or a glass transition temperature of greater than 40° C. can be chosen in particular from waxes, fillers, metal oxides, metal particles and their mixtures.

The wax under consideration in the context of the present invention is generally a lipophilic compound which is solid at ambient temperature (25° C.), which is or is not deformable, which exhibits a reversible solid/liquid change in state and which has a melting point of greater than or equal to 30° C. which can range up to 100° C. and in particular up to 90° C.

On bringing the wax to the liquid state (melting), it is possible to render it miscible with oils and to form a microscopically homogeneous mixture but, on bringing the temperature of the mixture back to ambient temperature, recrystallization of the wax in the oils of the mixture is obtained.

In particular, the waxes suitable for the invention can exhibit a melting point of greater than or equal to 45° C., better still of greater than or equal to 50° C. and even better still of greater than or equal to 60° C.

Within the meaning of the invention, the melting point corresponds to the temperature of the most endothermic peak observed by thermal analysis (DSC) as described in Standard ISO 11357-3; 1999. The melting point of the wax can be measured using a differential scanning calorimeter (DSC), for example the calorimeter sold under the name “MDSC 2920” by TA Instruments.

The measurement protocol is as follows:

A 5 mg sample of wax placed in a crucible is subjected to a first rise in temperature ranging from −20° C. to 100° C. at a heating rate of 10° C./minute, is then cooled from 100° C. to −20° C. at a cooling rate of 10° C./minute and, finally, is subjected to a second rise in temperature ranging from −20° C. to 100° C. at a heating rate of 5° C./minute. During the second rise in temperature, the variation in the difference in power absorbed by the empty crucible and by the crucible comprising the sample of wax is measured as a function of the temperature. The melting point of the compound is the value of the temperature corresponding to the tip of the peak of the curve representing the variation in the difference in power absorbed as a function of the temperature.

The waxes capable of being used in the compositions according to the invention are chosen from waxes of animal, vegetable, mineral or synthetic origin, and their mixtures, which are solid at ambient temperature.

The waxes which can be used in the compositions according to the invention generally exhibit a hardness ranging from 0.01 MPa to 15 MPa, in particular of greater than 0.05 MPa and especially of greater than 0.1 MPa.

The hardness is determined by the measurement of the compressive force measured at 20° C. using a texture analyser sold under the name TA-XT2 by Rheo, equipped with a stainless steel cylinder with a diameter of 2 mm which is displaced at the measuring rate of 0.1 mm/s and which penetrates the wax to a penetration depth of 0.3 mm.

The measurement protocol is as follows:

The wax is melted at a temperature equal to the melting point of the wax+10° C. The molten wax is cast in a receptacle with a diameter of 25 mm and a depth of 20 mm. The wax is recrystallized at ambient temperature (25° C.) for 24 hours, so that the surface of the wax is flat and smooth, and then the wax is stored at 20° C. for at least one hour before measuring the hardness or the tack.

The spindle of the texture analyser is displaced at a rate of 0.1 mm/s and then penetrates the wax to a penetration depth of 0.3 mm. When the spindle has penetrated the wax to the depth of 0.3 mm, the spindle is held stationary for 1 second (corresponding to the relaxation time) and is then withdrawn at the rate of 0.5 mm/s.

The value of the hardness is the maximum compressive force measured divided by the surface area of the cylinder of the texture analyser in contact with the wax.

Mention may in particular be made, by way of illustration of the waxes suitable for the invention, of hydrocarbon waxes, such as beeswax, lanolin wax and Chinese insect waxes; rice bran wax, carnauba wax, candelilla wax, ouricury wax, esparto wax, berry wax, shellac wax, Japan wax and sumac wax; montan wax, orange and lemon waxes, microcrystalline waxes, paraffin waxes and ozokerite; polyethylene waxes, the waxes obtained by the Fischer-Tropsch synthesis and waxy copolymers, and their esters, waxes obtained by catalytic hydrogenation of animal or vegetable oils having linear or branched C₈-C₃₂ fatty chains, such as isomerized jojoba oil, hydrogenated sunflower oil, hydrogenated castor oil, hydrogenated coconut oil, hydrogenated lanolin oil and di(1,1,1-trimethylolpropane)tetrastearate, sold under the name of Hest 2T-4S® by Heterene.

Mention may also be made of silicone waxes or fluorinated waxes.

Use may also be made of the waxes obtained by hydrogenation of castor oil esterified with cetyl alcohol which are sold under the names of Phytowax Castor 16L64® and 22L73® by Sophim. Such waxes are described in Application FR-A-2 792 190. Use may also be made of the trans-isomerized partially hydrogenated jojoba oil manufactured or sold by Desert Whale under the commercial reference Iso-Jojoba-50®.

Use may be made of a wax referred to as a “tacky wax”, that is to say having a tack of greater than or equal to 0.1 N.s and a hardness of less than or equal to 3.5 MPa.

Use may be made, as tacky wax, of a C₂₀-C₄₀ alkyl(hydroxystearyloxy)stearate (the alkyl group comprising from 20 to 40 carbon atoms), alone or as a mixture. Such a wax is sold in particular under the names “Kester Wax K 82 P®”, “Hydroxypolyester K 82 P®” and “Kester Wax K 80 P®” by Koster Keunen.

Use may also be made of waxes provided in the form of small particles having a size, expressed as volume-average “effective” diameter D[4,3], of the order of 0.5 to 30 micrometres, in particular of 1 to 20 micrometres and more particularly of 5 to 10 micrometres, subsequently denoted by the expression “microwaxes”.

The sizes of the particles can be measured by various techniques. Mention may in particular be made of light scattering techniques (dynamic and static), Coulter counter methods, measurements by rate of sedimentation (related to the size via Stokes' law) and microscopy. These techniques make it possible to measure a particle diameter and, for some of them, a particle size distribution.

Mention may in particular be made, as microwaxes which can be used in the compositions according to the invention, of carnauba microwaxes, such as that sold under the name of MicroCare 350® by Micro Powders, synthetic wax microwaxes, such as that sold under the name of MicroEase 114S® by Micro Powders, microwaxes composed of a mixture of carnauba wax and of polyethylene wax, such as those sold under the names of MicroCare 300® and 310® by Micro Powders, microwaxes composed of a mixture of carnauba wax and of synthetic wax, such as that sold under the name MicroCare 325® by Micro Powders, polyethylene microwaxes, such as those sold under the names of Micropoly 200®, 220®, 220L® and 250S® by Micro Powders, and polytetrafluoroethylene microwaxes, such as those sold under the names of Microslip 519® and 519 L® by Micro Powders.

Preferably, the first compound comprises at least one wax chosen from waxes exhibiting a melting point of greater than or equal to 60° C., preferably of greater than or equal to 70° C., such as carnauba wax, certain microcrystalline waxes, polyethylene waxes, rice bran wax, shellac wax and their mixtures.

The wax can represent from 0.1 to 50% by weight, with respect to the total weight of the first composition, preferably from 1 to 40% by weight and better still from 4 to 20% by weight.

The fillers can be chosen from those well known to a person skilled in the art and commonly used in cosmetic compositions.

The fillers can be inorganic or organic and lamellar or spherical. Mention may be made of talc, mica, silica, kaolin, powders formed of polyamide, such as Nylon®, sold under the name Orgasol® by Atochem, of poly-(3-alanine and of polyethylene, powders formed of tetrafluoroethylene polymers, such as Teflon®, starch, boron nitride, hollow polymer microspheres which are expanded, such as those of poly(vinylidene chloride)/acrylonitrile, for example those sold under the name Expancel® by Nobel Industrie, acrylic powders, such as those sold under the name Polytrap® by Dow Corning, particles formed of polymethyl methacrylate and silicone resin microbeads (Tospearls® from Toshiba, for example), precipitated calcium carbonate, magnesium carbonate, basic magnesium carbonate, hydroxyapatite, hollow silica microspheres (Silica Beads® from Maprecos), glass or ceramic microcapsules, metal soaps derived from organic carboxylic acids having from 8 to 22 carbon atoms and in particular from 12 to 18 carbon atoms, for example zinc stearate, magnesium stearate, lithium stearate, zinc laurate or magnesium myristate, heat-expandable particles, such as nonexpanded microspheres formed of vinylidene chloride/acrylonitrile/methyl methacrylate copolymer or of copolymer of homopolymer of acrylonitrile, such as, for example, those sold respectively under the references Expancel® 820 DU 40 and Expancel® 007WU by Akzo Nobel, and their mixtures.

According to one embodiment, the first compound comprises at least one filler chosen from powders formed of tetrafluoroethylene polymers.

The fillers can represent from 0.1 to 70% by weight, in particular from 1 to 60% by weight, indeed even from 5 to 20% by weight, with respect to the total weight of the first composition.

The metal particles and the metal oxides can be chosen from aluminium, iron oxide or titanium oxide particles, which may or may not be coated; such particles are mentioned later in the colouring materials.

Preferably, the first composition does not comprise a “compound capable of conferring, on the second composition, a dmax threading nature of greater than or equal to 5 mm” as defined later or comprises it in a content of less than or equal to 1% by weight, with respect to the weight of the first composition, preferably of less than or equal to 0.5% by weight and better still of less than or equal to 0.2% by weight.

II) Second Composition

1) Measurement of the Threading Nature

The threading nature of the second composition is determined using the texture analyser sold under the name TA-XT2i by Rheo, equipped with a temperature-controlled spindle, this spindle being a heating cartridge made of stainless steel with the reference Firerod DIV-STL (Watlow, France), with a diameter of 3.17 mm and with a length of 60 mm, with a maximum power of 40 W under a voltage of 24V, with a thermocouple of K loc C type.

The heating cartridge is supplied by a 5V/0.5 A LKS 005-5V direct current source from Elka-Electronique. Its temperature is regulated by a PID TC48 controller from Faucigny Instrument (France). An attachment lengthening piece was created in order to attach the temperature-controlled spindle to the measuring arm of the texture analyser.

The measurement is performed on threads of composition obtained by applying a vertical displacement on the spindle until contact with a sample of the composition and then, after a waiting period in contact, by applying a vertical upwards displacement on the spindle. If the composition has a hot threading nature, a thread is formed between the spindle in the withdrawal phase and the sample of the composition, the said thread becoming firmer under the effect of the cooling in the surrounding air. The dmax measurement consists of a measurement of the length of the threads thus formed after detachment from the surface of the spindle.

The protocol is as follows:
a) a sample of the composition is prepared by filling, to its maximum, a pan made of stainless steel with a depth of 2 mm and a diameter of 20 mm, the excess composition being levelled at the surface,
b) the temperature of spindle is controlled at 40° C.,
c) the spindle descends at a rate of 10 mm/s until contact with the surface of the composition,
d) the spindle is held stationary for 10 s and is then raised again at a rate of 10 mm/s.

During the phase of withdrawal of the spindle, a thread is formed between the composition and the spindle. As the spindle is moved away from the surface of the composition, the thread formed cools and becomes firmer. From a certain elongation, the thread detaches from the spindle.

The threading nature or dmax (expressed in mm) corresponds to the length of the thread obtained after breaking, measured with a graduated ruler.

The measurement of the threading nature is repeated three times for the same composition, at different points in the pan, and a dmax “threading” mean is calculated for each composition.

Stages b) to d) are repeated for the same composition at a fixed spindle temperature in stage b) of 50° C., 60° C., 70° C., 80° C., 90° C., 100° C., 110° C., 120° C., 130° C. and 140° C. respectively.

Among the threading values which may be obtained at the various temperatures, the highest value is retained as dmax threading nature value.

The second composition used in the method according to the invention exhibits a dmax threading nature of greater than or equal to 5 mm which can range up to 100 mm, preferably of greater than or equal to 7 mm, better still of greater than or equal to 10 mm and better still of greater than or equal to 15 mm.

Preferably, the second composition is capable of forming a thread such that, if, after formation of the thread and measurement of the dmax according to the protocol indicated above, the pan comprising the composition is placed vertically (so that the thread is in a horizontal position, that is to say subjected to gravity) for at least 30 seconds, the thread retains a minimum length of 5 mm (measurable manually with the graduated ruler).

The second composition exhibiting such a threading nature according to the invention makes it possible to obtain, when applied to keratinous fibres, a thread of composition in the extension of the eyelash. This thread retains its shape, remains rigid and does not shrink, which makes it possible to obtain an elongating effect on the eyelash.

The second composition employed in the method according to the invention is heated to a temperature of greater than or equal to 40° C., preferably of greater than or equal to 45° C., better still of greater than or equal to 50° C. and even better still of greater than or equal to 60° C.

The temperature can range up to 150° C., preferably up to 120° C., better still up to 100° C. and even better still up to 95° C.

Preferably, the second composition is brought to the temperature at which it exhibits the dmax threading nature, measured as indicated above (that is to say, to the temperature at which the threading nature is highest).

2) Compound Capable of Conferring, on the Second Composition, a Dmax Threading Nature of Greater Than or Equal to 5 mm

The composition advantageously comprises at least one compound which confers, on the said composition, a dmax threading nature of greater than or equal to 5 mm or a mixture of compounds such that the said mixture confers, on the said composition, a dmax threading nature of greater than or equal to 5 mm, when the composition is heated to a temperature of greater than or equal to 40° C.

This compound can be a hydrocarbon or silicone compound and advantageously exhibits a thermoplastic behaviour.

This compound is preferably solid at ambient temperature. Advantageously, it exhibits a dmax threading nature of greater than or equal to 5 mm when it is brought to a temperature of greater than or equal to 40° C., that is to say that it is capable of producing threads as described above, at a temperature of greater than or equal to 40° C., for example ranging from 40 to 150° C., preferably of greater than or equal to 45° C., for example ranging from 45 to 120° C., better still of greater than or equal to 50° C., for example ranging from 50 to 100° C., and even better still of greater than or equal to 60° C.

This compound is preferably a polymer and can advantageously be chosen from:

A/ Polymers and copolymers comprising at least one alkene monomer, in particular ethylene-based copolymers.

Such compounds can be chosen from:
copolymers of alkene and of vinyl acetate, in particular copolymers of ethylene and of vinyl acetate. Use is made in particular of the copolymers of ethylene and of vinyl acetate preferably comprising more than 25% by weight of vinyl acetate, with respect to the total weight of the polymer, for example from 25 to 50% by weight, better still from 25 to 35% by weight, for example of the order of 28% by weight, of vinyl acetate.

Mention is made, as example of ethylene/vinyl acetate copolymers, of those which are sold under the Elvax name by Du Pont de Nemours and in particular the compounds Elvax 40W, Elvax 140W, Elvax 200W, Elvax 205W, Elvax 210W and Elvax 310.

Mention may also be made of the products sold under the Evatane name by Arkema, such as Evatane 28-800. Mention may also be made of Melthene-H Grade H-6410M, provided by Tosoh Polymer.
copolymers of ethylene and of octene, such as, for example, the products sold under the “Affinity” reference by Dow Plastics, such as, for example, Affinity GA 1900 and GA 1950.

These polymers and copolymers can be used alone or as a mixture with at least one compound chosen from “tackifying” resins, such as described in the Handbook of Pressure Sensitive Adhesives, edited by Donatas Satas, 3rd ed., 1989, pp. 609-619, waxes, such as described later, and their combinations. The tackifying resins can in particular be chosen from rosin, rosin derivatives, hydrocarbon resins and their mixtures. Mention may in particular be made of indene hydrocarbon resins, such as the resins resulting from the polymerization predominantly of indene monomer with a minor proportion of monomers chosen from styrene, methylindene, methylstyrene and their mixtures. These resins can optionally be hydrogenated. They can exhibit a molecular weight ranging from 290 to 1150. Mention may in particular be made, as examples of indene resins, of the hydrogenated indene/methylstyrene/styrene copolymers sold under the “Regalite” name by Eastman Chemical, in particular Regalite R 1100, Regalite R 1090, Regalite R-7100, Regalite R1010 Hydrocarbon Resin and Regalite R1125 Hydrocarbon Resin.

Mention may be made, as mixture based on ethylene/vinyl acetate copolymer, for example of the products sold under the Coolbind name by National Starch.

These polymers can be provided in their pure form or can be conveyed in an aqueous phase or an organic solvent phase.

B/ Polyvinyl acetate homopolymers, preferably exhibiting a molecular weight of less than 20 000, such as, for example, Raviflex BL1S from Vinavil.

C/ Silicone resins

These resins are crosslinked organosiloxane polymers. The nomenclature of silicone resins is known under the “name” of “MDTQ”, the resin being described according to the various monomeric siloxane units which it comprises, each of the “MDTQ” letters characterizing the type of unit.

The letter M represents the monofunctional unit of formula (CH₃)₃SiO_{1/2}, the silicon atom being connected to a single oxygen atom in the polymer comprising this unit.

The letter D means a difunctional unit (CH₃)₂SiO_{2/2} in which the silicon atom is connected to two oxygen atoms. The letter T represents a trifunctional unit of formula (CH₃) SiO_{3/2}.

In the M, D and T units defined above, at least one of the methyl groups can be replaced by a group R which is different from the methyl group, such as a hydrocarbon (in particular alkyl) radical having from 2 to 10 carbon atoms or a phenyl group or alternatively a hydroxyl group.

Finally, the letter Q means a tetrafunctional unit SiO_{4/2} in which the silicon atom is bonded to four oxygen atoms, themselves bonded to the remainder of the polymer.

Mention is made in particular of T resins, especially functionalized T silicone resins, such as polyphenylsiloxanes, especially functionalized by silanol (Si—OH) groups, such as that sold under the reference Dow Corning (R) Z-1806.

D/ Film-forming block ethylenic polymers

These polymers preferably comprise at least one first block and at least one second block having different glass transition temperatures (Tg), the said first and second blocks being connected to one another via an intermediate block comprising at least one constituent monomer of the first block and at least one constituent monomer of the second block.

Advantageously, the first and second blocks of the block polymer are incompatible with one another.

Such polymers are described, for example, in the documents EP 1 411 069 or WO 04/028488 or WO 04/028493.

The term “block” polymer is understood to mean a polymer comprising at least 2 distinct blocks, for example at least 3 distinct blocks.

The first and second blocks of the polymer differ from one another in their degree of deformability. Thus, the first block can be rigid and the second block can be flexible.

The glass transition temperatures of the flexible and rigid blocks can be theoretical Tg values determined from the theoretical Tg values of the constituent monomers of each of the blocks, which can be found in a reference handbook, such as the Polymer Handbook, 3^{rd} ed., 1989, John Wiley, according to the following relationship, referred to as the Fox Law:

$$1 / Tg = {\sum }_{i} ( {ω}_{i}^{′} / {Tg}_{i} ) ,$$

\acute{ω}ᵢ being the mass fraction of the monomer i in the block under consideration and Tgᵢ being the glass transition temperature of the homopolymer of the monomer i.

Unless otherwise indicated, the Tg values indicated for the first and second blocks in the present patent application are theoretical Tg values.

The rigid block can have a Tg of greater than 20° C.

The flexible block can have a Tg of less than or equal to 20° C.

According to an embodiment, the copolymer comprises a first rigid block and a second flexible block.

Preferably, the proportion of the rigid block ranges from 20 to 90% by weight of the copolymer, better still from 30 to 90% by weight and even better still from 50 to 90% by weight.

Preferably, the proportion of the flexible block ranges from 5 to 75% by weight of the copolymer, preferably from 10 to 50% by weight and better still from 15 to 45% by weight.

Rigid Block

In the context of the present invention, the rigid block or blocks are more particularly formed from the following monomers:
methacrylates of formula CH₂═C(CH₂)—COOR₁ in which R₁ represents an unsubstituted linear or branched C₁ to C₄ alkyl group, such as a methyl, ethyl, propyl or isobutyl group, or R₁ represents a C₄ to C₁₂ cycloalkyl group, such as an isobornyl group,
acrylates of formula CH₂═CH—COOR₂ in which R₂ represents a tert-butyl group or a C₄ to C₁₂ cycloalkyl group, such as an isobornyl group,
(meth)acrylamides of formula:

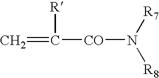

where R₇ and R₈, which are identical or different, each represent a hydrogen atom or a linear or branched C₁ to C₁₂ alkyl group, such as n-butyl, t-butyl, isopropyl, isohexyl, isooctyl or isononyl group, or R₇ represents H and R₈ represents a 1,1-dimethyl-3-oxobutyl group,
and R′ denotes H or methyl.

Mention may be made, as example of monomers of this type, of N-butylacrylamide, N-(t-butyl)acrylamide,

N-isopropylacrylamide, N,N-dimethylacrylamide and N,N-dibutylacrylamide,
and their mixtures.

Particularly preferred monomers of the rigid block are isobornyl methacrylate, isobornyl acrylate and their mixtures.

Flexible Block

In the context of the present invention, the flexible block or blocks are more particularly formed from the following monomers:
acrylates of formula CH₂═CHCOOR₃, with R₃ representing an unsubstituted linear or branched C₁ to C₁₂ alkyl group, such as an isobutyl group (with the exception of a tert-butyl group), in which one or more heteroatoms chosen from O, N and S is/are optionally intercalated,
methacrylates of formula CH₂═C(CH₃)—COOR₄, with

R₄ representing an unsubstituted linear or branched C₆ to C₁₂ alkyl group in which one or more heteroatoms chosen from O, N and S is/are optionally intercalated;
vinyl esters of formula R₅—CO—O—CH═CH₂ where R₅ represents a linear or branched C₄ to C₁₂ alkyl group;
C₄ to C₁₂ alkyl vinyl ethers;
and their mixtures.

Particularly preferred monomers of the flexible block are isobutyl acrylate.

Each of the blocks can comprise a minor proportion of at least one constituent monomer of the other block.

Thus, the first block can comprise at least one constituent monomer of the second block, and vice versa. Each of the first and/or second blocks can comprise, in addition to the monomers indicated above, one or more other monomers, known as additional monomers, which are different from the main monomers mentioned above. This additional monomer is chosen for example, from:

a) hydrophilic monomers, such as:
monomers possessing ethylenic unsaturation(s), other than acrylic acid, comprising at least one carboxylic or sulphonic acid functional group, such as, for example, methacrylic acid, crotonic acid, maleic anhydride, itaconic acid, fumaric acid, maleic acid, acrylamidopropanesulphonic acid, vinylbenzoic acid or vinylphosphonic acid, and the salts of these,
monomers possessing ethylenic unsaturation(s) comprising at least one tertiary amine functional group, such as 2-vinylpyridine, 4-vinylpyridine, dimethylaminoethyl methacrylate, diethylaminoethyl methacrylate, dimethylaminopropyl methacrylamide and the salts of these,
methacrylates of formula CH₂═C(CH₃)—COOR₆

in which R₆ represents a linear or branched alkyl group comprising from 1 to 4 carbon atoms, such as a methyl, ethyl, propyl or isobutyl group, the said alkyl group being substituted by one or more substituents chosen from hydroxyl groups (such as 2-hydroxypropyl methacrylate or 2-hydroxyethyl methacrylate) and halogen atoms (Cl, Br, I, F), such as trifluoroethyl methacrylate,
methacrylates of formula CH₂═C(CH₃)—COOR₉,

R₉ representing a linear or branched C₆ to C₁₂ alkyl group in which one or more heteroatoms chosen from O, N and S is/are optionally intercalated, the said alkyl group being substituted by one or more substituents chosen from hydroxyl groups and halogen atoms (Cl, Br, I, F);
acrylates of formula CH₂═CHCOOR₁₀,

R₁₀ representing a linear or branched C₁ to C₁₂ alkyl group substituted by one or more substituents chosen from hydroxyl groups and halogen atoms (Cl, Br, I and F), such as 2-hydroxypropyl acrylate and 2-hydroxyethyl acrylate, or R₁₀ representing a (C₁-C₁₂)alkyl-O-POE (polyoxyethylene) with repetition of the oxyethylene unit from 5 to 30 times, for example methoxy-POE, or R₁₀ representing a polyoxyethylene group comprising from 5 to 30 ethylene oxide units,

b) monomers possessing ethylenic unsaturation(s) comprising one or more silicon atoms, such as methacryloyloxypropyl-trimethoxysilane, or methacryloyloxypropyltris(trimethylsiloxy)silane,
and their mixtures.

This or these additional monomers generally represent(s) an amount of less than or equal to 30% by weight, for example from 1 to 30% by weight, preferably from 5 to 20% by weight and more preferably from 7 to 15% by weight, of the total weight of the first and/or second blocks.

According to one embodiment, the copolymer can comprise at least one first block and at least one second block connected to one another via an intermediate segment comprising at least one constituent monomer of the first block and at least one constituent monomer of the second block.

Preferably, the intermediate block results essentially from constituent monomers of the first block and of the second block.

Advantageously, the intermediate segment comprising at least one constituent monomer of the first block and at least one constituent monomer of the second block of the copolymer is a random polymer.

Advantageously, the copolymer results essentially from monomers chosen from alkyl methacrylates, alkyl acrylates and their mixtures.

The term “essentially” is understood to mean, in that which precedes and in that which follows, comprising at least 85%, preferably at least 90%, better still at least 95% and even better still 100%.

As regards the acrylate and methacrylate esters, they can derive from the esterification of linear or branched, cyclic or aromatic C₁ to C₁₂ alcohols, in particular C₄ to C₁₀ alcohols.

Mention may in particular be made, by way of illustration and without implied limitation of these alcohols, of isoborneol.

According to one embodiment, the said copolymer comprises at least acrylate and methacrylate monomers deriving from the esterification of the same alcohol and in particular isoborneol.

Preferably, the film-forming linear block polymer comprises at least isobornyl acrylate monomers, at least isobornyl methacrylate monomers and at least isobutyl acrylate monomers.

According to an alternative embodiment, the block polymer can comprise at least:
a rigid block, which is an isobornyl methacrylate/isobornyl acrylate copolymer, and
a flexible block, which is an isobutyl acrylate copolymer.

More specifically, the copolymer can comprise from 50 to 80% by weight of isobornyl methacrylate/acrylate and from 10 to 20% by weight of isobutyl acrylate.

The weight-average molecular weight (Mw) of the copolymer preferably ranges from 80 000 to 300 000, indeed even from 100 000 to 150 000.

The number-average molecular weight (Mn) of the copolymer preferably ranges from 20 000 to 90 000; for example it ranges from 25 000 to 45 000.

E/ Copolymers of dienes and of styrene, in particular copolymers of butadiene and styrene.

Mention may in particular be made of the styrene/butadiene copolymers sold under the Pliolite S5E reference by Eliokem.

F/ Polyesters comprising at least one monomer carrying at least one —SO₃M group (M representing a hydrogen atom, an ammonium ion NH₄⁺ or a metal ion), also known as sulphopolyesters.

These polyesters advantageously have a glass transition temperature (Tg) of greater than 38° C.

They can exhibit a weight-average molecular weight advantageously of less than 200 000, for example ranging from 10 000 to 50 000.

These polyesters can be obtained in a known way by polycondensation with at least one dicarboxylic acid with at least one polyol, in particular diols. The dicarboxylic acid can be aliphatic, alicyclic or aromatic. Mention may be made, as examples of such acids, of: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexane-dicarboxylic acid, isophthalic acid, terephthalic acid, 2,5-norbornanedicarboxylic acid, diglycolic acid, thiodipropionic acid, 2,5-naphthalenedicarboxylic acid or 2,6-naphthalenedicarboxylic acid. These dicarboxylic acid monomers can be used alone or as a combination of at least two dicarboxylic acid monomers. The choice is preferably made, among these monomers, of phthalic acid, isophthalic acid or terephthalic acid.

The diol can be chosen from aliphatic, alicyclic or aromatic diols. Use is preferably made of a diol chosen from: ethylene glycol, diethylene glycol, triethylene glycol, 1,3-propanediol, cyclohexanedimethanol or 4-butanediol.

Use may be made, as other polyols, of glycerol, pentaerythritol, sorbitol or trimethylolpropane.

Polyesteramides can be obtained analogously to the polyesters by polycondensation of diacids with diamines or aminoalcohols. Use may be made, as diamines, of ethylenediamine, hexamethylenediamine, meta-phenylenediamine or para-phenylenediamine. Use may be made, as aminoalcohol, of monoethanolamine.

The polyester comprises at least one monomer carrying at least one —SO₃M group, with M representing a hydrogen atom, an ammonium ion NH₄⁺ or a metal ion, such as, for example, an Na⁺, Li⁺, K⁺, Mg²⁺, Ca²⁺, Cu²⁺, Fe²⁺ or Fe³⁺ ion. Use may in particular be made of a bifunctional aromatic monomer comprising such an —SO₃M group.

The aromatic ring system of the bifunctional aromatic monomer additionally carrying an —SO₃M group as described above can be chosen, for example, from the benzene, naphthalene, anthracene, biphenyl, oxydiphenyl, sulphonyldiphenyl or methylenediphenyl ring systems. Mention may be made, as example of bifunctional aromatic monomer additionally carrying an —SO₃M group, of: sulphoisophthalic acid, sulphoterephthalic acid, sulphophthalic acid or 4-sulphonaphthalene-2,7-dicarboxylic acid.

It is preferable to use copolymers based on isophthalate/sulphoisophthalate and more particularly copolymers obtained by condensation of diethylene glycol, cyclohexanedimethanol, isophthalic acid and sulphoisophthalic acid.

Such polymers are sold, for example, under the Eastman AQ® trade name by Noveon, such as, for example, Eastman AQ 38S.

G/ Waxes

The waxes can be chosen from the waxes described above for the first composition.

In particular, the waxes are chosen from paraffin wax.

H/ Fibres

The term “fibre” should be understood as meaning an object with a length L and a diameter D such that L is much greater than D, D being the diameter of the circle in which the cross section of the fibre is framed. In particular, the L/D ratio (or aspect ratio) is chosen within the range from 3.5 to 2500, in particular from 5 to 500 and more particularly from 5 to 150.

The fibres which can be used in the composition of the invention can be fibres of synthetic or natural and inorganic or organic origin. They can be short or long, individual or organized, for example plaited, and hollow or solid. They can have any shape and can in particular be circular or polygonal (square, hexagonal or octagonal) in cross section, according to the specific application envisaged. In particular, their ends are blunted and/or polished to prevent injury.

In particular, the fibres have a length ranging from 1 μm to 10 mm, in particular from 0.1 mm to 5 mm and more particularly from 0.3 mm to 3.5 mm. Their cross section can be included within a circle with a diameter ranging from 2 nm to 500 μm, in particular ranging from 100 nm to 100 μm and more particularly ranging from 1 μm to 50 μm. The weight or count of the fibres is often given in denier or decitex and represents the weight in grams per 9 km of yarn. The fibres according to the invention can in particular have a count chosen within the range from 0.15 to 30 denier and in particular from 0.18 to 18 denier.

The fibres which can be used in the composition of the invention can be chosen from rigid or nonrigid fibres.

They can be of synthetic or natural and inorganic or organic origin.

Furthermore, the fibres may or may not be surface treated, may or may not be coated and may or may not be coloured.

Mention may be made, as fibres which can be used in the composition according to the invention, of fibres which are not rigid, such as polyamide (Nylon®) fibres, or fibres which are rigid, such as polyimideamide fibres, for example those sold under the Kermel® or Kermel Tech® names by Rhodia, or poly(p-phenylene terephthalamide) (or aramid) fibres, sold in particular under the Kevlar® name by DuPont de Nemours.

According to one embodiment, the composition comprises, as compounds which confer, on the composition, a dmax threading nature of greater than or equal to 5 mm, at least a mixture of wax, in particular paraffin wax, especially in a content of greater than or equal to 90% by weight, with respect to the total weight of the composition, and fibres, especially cellulose fibres, for example in a content of 1 to 5% by weight, with respect to the total weight of the composition.

According to an advantageous embodiment, the second composition comprises at least one ethylene/vinyl acetate copolymer.

According to one embodiment, the composition comprises, as compounds which confer, on the composition, a dmax threading nature of greater than or equal to 5 mm, a mixture of wax, in particular of paraffin wax, and of ethylene/vinyl acetate copolymer.

The said mixture can comprise in particular from 50 to 65% by weight of ethylene/vinyl acetate copolymer, with respect to the total weight of the mixture, and from 35 to 50% by weight of paraffin wax, with respect to the total weight of the mixture.

Preferably, the ethylene/vinyl acetate copolymer comprises more than 25% by weight, with respect to the total weight of the polymer, of vinyl acetate, for example approximately 28% by weight.

Preferably, the ethylene/vinyl acetate copolymer exhibits a weight-average molecular weight (Mw) ranging from 50 000 to 80 000, better still from 60 000 to 70 000 and even better still from 63 000 to 73 000.

The compound or the mixture of compounds which confers, on the composition, a dmax threading nature of greater than or equal to 5 mm can be present in the composition in a content of dry matter of at least 5% by weight, with respect to the total weight of the composition, for example ranging from 5 to 100% by weight, with respect to the total weight of the composition, preferably ranging from 10 to 100% by weight and better still from 12 to 100% by weight.

III/ Aqueous Phase

The first and/or the second composition according to the invention can preferably comprise an aqueous medium, constituting an aqueous phase, which can form the continuous phase of the composition.

The aqueous phase of the composition or compositions according to the invention is advantageously a continuous aqueous phase.

The term “composition comprising a “continuous” aqueous phase” is understood to mean that the composition exhibits a conductivity, measured at 25° C., of greater than 23 μS/cm (microSiemens/cm), the conductivity being measured, for example, using an MPC227 conductivity meter from Mettler Toledo and an Inlab730 conductivity measurement cell. The measurement cell is immersed in the composition, so as to remove the air bubbles capable of being formed between the 2 electrodes of the cell. The conductivity is read as soon as the value of the conductivity meter has stabilized. A mean is taken over at least 3 successive measurements.

The aqueous phase can be composed essentially of water; it can also comprise a mixture of water and of water-miscible solvent (miscibility in water of greater than 50% by weight at 25° C.), such as low monoalcohols having from 1 to 5 carbon atoms, such as ethanol or isopropanol, glycols having from 2 to 8 carbon atoms, such as propylene glycol, ethylene glycol, 1,3-butylene glycol or dipropylene glycol, C₃-C₄ ketones, C₂-C₄ aldehydes and their mixtures.

The aqueous phase (water and optionally the water-miscible solvent) can be present in a content ranging from 1% to 95% by weight, with respect to the total weight of the composition comprising it, preferably ranging from 3% to 80% by weight and preferentially ranging from 5% to 60% by weight.

Preferably, the first composition comprises an aqueous continuous phase.

According to one embodiment, the second composition comprises less than 20% by weight of water, with respect to the total weight of the composition, preferably less than 10% by weight and better still less than 5% by weight. It can be devoid of water (anhydrous).

Emulsifying System

The compositions according to the invention can comprise emulsifying surface-active agents present in particular in a proportion ranging from 0.1% to 20% and better still from 0.3% to 15% by weight, with respect to the total weight of each composition comprising them.

According to the invention, use is generally made of an emulsifier appropriately chosen in order to obtain an oil-in-water emulsion. Use may in particular be made of an emulsifier having, at 25° C., an HLB (hydrophilic-lipophilic balance) balance within the meaning of Griffin of greater than or equal to 8.

The HLB value according to Griffin is defined in J. Soc. Cosm. Chem., 1954 (volume 5), pages 249-256.

These surface-active agents can be chosen from nonionic, anionic, cationic or amphoteric surface-active agents or also surface-active emulsifiers. Reference may be made to the document “Encyclopedia of Chemical Technology, Kirk-Othmer”, volume 22, pp. 333-432, 3rd edition, 1979, Wiley, for the definition of the properties and functions (emulsifying) of surfactants, in particular pp. 347-377 of this reference for the anionic, amphoteric and nonionic surfactants.

The surfactants preferentially used in the compositions according to the invention are chosen from:
a) nonionic surface-active agents with an HLB of greater than or equal to 8 to 25° C., used alone or as a mixture; mention may in particular be made of:
oxyethylenated and/or oxypropylenated ethers (which can comprise from 1 to 150 oxyethylene and/or oxypropylene groups) of glycerol;
oxyethylenated and/or oxypropylenated ethers (which can comprise from 1 to 150 oxyethylene and/or oxypropylene groups) of fatty alcohols (in particular of C₈-C₂₄ and preferably C₁₂-C₁₈ alcohols), such as the oxyethylenated ether of stearyl alcohol comprising 20 oxyethylene groups (CTFA name “Steareth-20”), such as Brij 78, sold by Uniqema, the oxyethylenated ether of cetearyl alcohol comprising 30 oxyethylene groups (CTFA name “Ceteareth-30”) and the oxyethylenated ether of the mixture of C₁₂-C₁₅ fatty alcohols comprising 7 oxyethylene groups (CTFA name “C12-15 Pareth-7”) sold under the name Neodol 25-7® by Shell Chemicals,
esters of fatty acid (in particular of C₈-C₂₄ and preferably C₁₆-C₂₂ acid) and of polyethylene glycol (which can comprise from 1 to 150 ethylene glycol units), such as PEG-50 stearate and PEG-40 monostearate, sold under the name Myrj 52P® by ICI Uniqema,
esters of fatty acid (in particular of C₈-C₂₄ and preferably C₁₆-C₂₂ acid) and of the oxyethylenated and/or oxypropylenated glycerol ethers (which can comprise from 1 to 150 oxyethylene and/or oxypropylene groups), such as PEG-200 glyceryl monostearate, sold under the name Simulsol 220 TM® by SEPPIC; polyethoxylated glyceryl stearate comprising 30 ethylene oxide groups, such as the product Tagat S® sold by Goldschmidt, polyethoxylated glyceryl oleate comprising 30 ethylene oxide groups, such as the product Tagat O® sold by Goldschmidt, polyethoxylated glyceryl cocoate comprising 30 ethylene oxide groups, such as the product Varionic LI 13® sold by Sherex, polyethoxylated glyceryl isostearate comprising 30 ethylene oxide groups, such as the product Tagat L® sold by Goldschmidt and polyethoxylated glyceryl laurate comprising 30 ethylene oxide groups, such as the product Tagat I® from Goldschmidt,
esters of fatty acid (in particular of C₈-C₂₄ and preferably C₁₆-C₂₂ acid) and of the oxyethylenated and/or oxypropylenated sorbitol ethers (which can comprise from 1 to 150 oxyethylene and/or oxypropylene groups), such as polysorbate 60, sold under the name Tween 60® by Uniqema,
dimethicone copolyol, such as that sold under the name Q2-5220® by Dow Corning,
dimethicone copolyol benzoate (Finsolv SLB 1010 and 201® from Fintex),
copolymers of propylene oxide and of ethylene oxide, also known as EO/PO polycondensates,
and their mixtures.

The EO/PO polycondensates are more particularly copolymers consisting of polyethylene glycol and polypropylene glycol seals, such as, for example, polyethylene glycol/polypropylene glycol/polyethylene glycol triblock polycondensates. These triblock polycondensates have, for example, the following chemical structure:

H—(O—CH₂—CH₂)ₐ—(O—CH(CH₃)—CH₂)_{b}—(O—CH₂—CH₂)ₐ—OH,
in which formula a ranges from 2 to 120 and b ranges from 1 to 100.

The EO/PO polycondensate preferably has a weight-average molecular weight ranging from 1000 to 15 000 and better still ranging from 2000 to 13 000. Advantageously, the said EO/PO polycondensate has a cloud point, at 10 g/l in distilled water, of greater than or equal to 20° C., preferably of greater than or equal to 60° C. The cloud point is measured according to the standard ISO 1065. Mention may be made, as EO/PO polycondensate which can be used according to the invention, of the polyethylene glycol/polypropylene glycol/polyethylene glycol triblock polycondensates sold under the Synperonic® names, such as Synperonic PE/L44® and Synperonic PE/F127®, by ICI,
b) nonionic surface-active agents with an HLB of less than 8 at 25° C., optionally in combination with one or more nonionic surface-active agents with an HLB of greater than 8 at 25° C., such as mentioned above, such as:
esters and ethers of monosaccharides, such as sucrose mixtures, such as Arlatone 2121®, sold by ICI, or Span 65V, from Uniqema;
esters of fatty acids (in particular of C₈-C₂₄ and preferably C₁₆-C₂₂ acid) and of polyol, in particular of glycerol or of sorbitol, such as glyceryl stearate, such as the product sold under the name Tegin M® by Goldschmidt, glyceryl laurate, such as the product sold under the name Imwitor 312® by Hüls, polyglyceryl-2 stearate, sorbitan tristearate or glyceryl ricinoleate;
oxyethylenated and/or oxypropylenated ethers, such as the oxyethylenated ether of stearyl alcohol comprising 2 oxyethylene groups (CTFA name “Steareth-2”), such as Brij 72, sold by Uniqema;
the cyclomethicone/dimethicone copolyol mixture sold under the name Q2-3225C® by Dow Corning,
c) anionic surfactants, such as:
salts of polyoxyethylenated fatty acids, in particular those derived from amines or the alkali metal salts, and their mixtures;
phosphoric esters and their salts, such as “DEA oleth-10 phosphate” (Crodafos N 10N from Croda) or monopotassium monocetyl phosphate (Amphisol K from Givaudan or Arlatone MAP 160K from Uniqema);
sulphosuccinates, such as “Disodium PEG-5 citrate lauryl sulphosuccinate” and “Disodium ricinoleamido MEA sulphosuccinate”;
alkyl ether sulphates, such as sodium lauryl ether sulphate;
isethionates;
acylglutamates, such as “Disodium hydrogenated tallow glutamate” (Amisoft HS-21 R®, sold by Ajinomoto), and their mixtures.

Mention may in particular be made, by way of representation of cationic surfactants, of:
alkyl imidazolidiniums, such as isostearyl ethylimidonium ethosulphate,
ammonium salts, such as N,N,N-trimethyl-1-docosanaminium chloride (behentrimonium chloride).

The compositions according to the invention can also comprise one or more amphoteric surfactants, such as N-acylamino acids, for example N-acylaminoacetates and disodium cocoamphodiacetate, and amine oxides, such as stearamine oxide, or also silicone surfactants, such as dimethicone copolyol phosphates, such as that sold under the name Pecosil PS 100® by Phoenix Chemical.

According to one embodiment, the compositions according to the invention, in particular the first composition, comprise, as emulsifying system, the following combination:
of at least one C₁₀-C₃₀ alkyl phosphate surface-active agent, and
of at least one ether of C₈-C₂₄ fatty alcohol and of polyethylene glycol, the said ether comprising from 1 to 19 oxyethylene units and exhibiting an HLB<8 at 25° C.

According to one embodiment, the said emulsifying system can additionally comprise at least one ether of C₈-C₂₄ fatty alcohol and of polyethylene glycol, the said ether comprising from 20 to 1000 oxyethylene units and with an HLB>8 at 25° C., and at least one fatty alcohol comprising from 10 to 30 carbon atoms.

According to an advantageous embodiment, the cosmetic compositions according to the present invention, in particular the first composition, comprise less than 1% by weight, preferably less than 0.5% by weight, with respect to the total weight of the composition, of triethanolamine and better still are devoid of triethanolamine.

According to an advantageous alternative form, the cosmetic compositions according to the invention, in particular the first composition, comprise less than 1% by weight, preferably less than 0.5% by weight, with respect to the total weight of the composition, of triethanolamine stearate and better still are devoid of triethanolamine stearate.

Water-Soluble Gelling Agent

The first and/or the second composition according to the invention can comprise a water-soluble gelling agent.

The water-soluble gelling agents which can be used in the compositions according to the invention can be chosen from:
homo- or copolymers of acrylic acid or methacrylic acid or their salts and their esters and in particular the products sold under the names Versicol F® or Versicol K® by Allied Colloid, Ultrahold 8® by Ciba-Geigy, poly(acrylic acid)s of Synthalen K type,
copolymers of acrylic acid and of acrylamide, sold in the form of their sodium salts under the Reten® names by Hercules, poly(sodium methacrylate), sold under the name Darvan No. 7® by Vanderbilt, sodium salts of poly(hydroxycarboxylic acid)s, sold under the name Hydagen F® by Henkel,
copolymers of poly(acrylic acid)s and of alkyl acrylates of Pemulen type,
AMPS (poly(acrylamidomethylpropanesulphonic acid) partially neutralized with aqueous ammonia and highly crosslinked), sold by Clariant,
AMPS/acrylamide copolymers of Sepigel® or Simugel® type, sold by SEPPIC, and
copolymers of AMPS and of alkyl methacrylates which are polyoxyethylenated (crosslinked or noncrosslinked),
proteins, such as proteins of plant origin, such as wheat or soya proteins; proteins of animal origin, such as keratins, for example keratin hydrolysates and sulphonic keratins;
cellulose polymers, such as hydroxyethylcellulose, hydroxypropylcellulose, methylcellulose, ethylhydroxy-ethylcellulose, carboxymethylcellulose and quaternized cellulose derivatives;
acrylic polymers or copolymers, such as polyacrylates or polymethacrylates;
vinyl polymers, such as polyvinylpyrrolidones, copolymers of methyl vinyl ether and of malic anhydride, the copolymer of vinyl acetate and of crotonic acid, copolymers of vinylpyrrolidone and of vinyl acetate, copolymers of vinylpyrrolidone and of caprolactam, or poly(vinyl alcohol);
optionally modified polymers of natural origin, such as:
gums arabic, guar gum, xanthan derivatives or karaya gum;
alginates and carrageenans;
glycoaminoglycans, hyaluronic acid and its derivatives;
shellac resin, gum sandarac, dammars, elemis or copals;
deoxyribonucleic acid;
mucopolysaccharides, such as chondroitin sulphates,
and their mixtures.

Some of these water-soluble gelling agents can also act as film-forming polymers.

According to a preferred embodiment, the first and/or the second composition comprises at least one AMPS/acrylamide copolymer.

The water-soluble gelling polymer can be present in the composition comprising it in a content of dry matter ranging from 0.01% to 60% by weight, preferably from 0.5% to 40% by weight, better still from 1% to 30% by weight, indeed even from 5% to 20% by weight, with respect to the total weight of the composition comprising it.

Oils

The first and/or the second composition according to the invention can additionally comprise one or more nonaqueous oils or fatty substances which are liquid at ambient temperature (25° C.) and atmospheric pressure (760 mmHg).

The oil can be chosen from volatile oils and/or nonvolatile oils, and their mixtures.

The oil or oils can be present in a content ranging from 0.1% to 95% by weight, preferably from 0.5% to 60% by weight, with respect to the total weight of the composition comprising them.

The term “volatile oil” is understood to mean, within the meaning of the invention, an oil capable of evaporating on contact with the keratinous fibres in less than one hour at ambient temperature and atmospheric pressure. The volatile organic solvent or solvents and the volatile oils of the invention are volatile cosmetic organic solvents and oils which are liquid at ambient temperature and which have a nonzero vapour pressure, at ambient temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The term “nonvolatile oil” is understood to mean an oil which remains on the keratinous fibre at ambient temperature and atmospheric pressure for at least several hours and which has in particular a vapour pressure of less than 10⁻³ mmHg (0.13 Pa).

These oils can be hydrocarbon oils, silicone oils, fluorinated oils or their mixtures.

The term “hydrocarbon oil” is understood to mean an oil comprising mainly hydrogen and carbon atoms and optionally oxygen, nitrogen, sulphur and phosphorus atoms. Volatile hydrocarbon oils can be chosen from hydrocarbon oils having from 8 to 16 carbon atoms, in particular branched C₈-C₁₆ alkanes, such as C₈-C₁₆ isoalkanes of petroleum origin (also known as isoparaffins), such as isododecane (also known as 2,2,4,4,6-pentamethylheptane), isodecane or isohexadecane, for example the oils sold under the Isopar or Permethyl trade names, branched C₈-C₁₆ esters, isohexyl neopentanoate, and their mixtures. Other volatile hydrocarbon oils, such as petroleum distillates, in particular those sold under the Shell Solt name by Shell, can also be used. Preferably, the volatile solvent is chosen from volatile hydrocarbon oils having from 8 to 16 carbon atoms and their mixtures.

Use may also be made, as volatile oils, of volatile silicones, such as, for example, volatile linear or cyclic silicone oils, in particular those having a viscosity 8 centistokes (8×10⁻⁶ m²/s) and having in particular from 2 to 7 silicon atoms, these silicones optionally comprising alkyl or alkoxy groups having from 1 to 10 carbon atoms. Mention may in particular be made, as volatile silicone oil which can be used in the invention, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

Mention may also be made of the volatile linear alkyltrisiloxane oils of general formula (I)

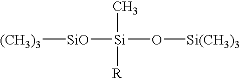

where R represents an alkyl group comprising from 2 to 4 carbon atoms, one or more hydrogen atoms of which can be substituted by a fluorine or chlorine atom.

Mention may be made, among the oils of general formula (I), of:

3-butyl-1,1,1,3,5,5,5-heptamethyltrisiloxane,

3-propyl-1,1,1,3,5,5,5-heptamethyltrisiloxane, and

3-ethyl-1,1,1,3,5,5,5-heptamethyltrisiloxane,

corresponding to the oils of formula (I) for which R is respectively a butyl group, a propyl group or an ethyl group. Use may also be made of volatile fluorinated solvents, such as nonafluoromethoxybutane or perfluoromethylcyclopentane.

The first and/or the second composition can also comprise at least one nonvolatile oil, chosen in particular from nonvolatile hydrocarbon oils and/or silicone oils and/or fluorinated oils.

Mention may in particular be made, as nonvolatile hydrocarbon oil, of:
hydrocarbon oils of vegetable origin, such as triesters of fatty acids and of glycerol, the fatty acids of which can have varied chain lengths from C₄ to C₂₄, it being possible for these chains to be linear or branched and saturated or unsaturated; these oils are in particular wheat germ, sunflower, grape seed, sesame, maize, apricot kernel, castor, Shea, avocado, olive, soybean, sweet almond, palm, rapeseed, cottonseed, hazelnut, macadamia, jojoba, alfalfa, poppy, pumpkinseed, cucumber, blackcurrant seed, evening primrose, millet, barley, quinoa, rye, safflower, candlenut, passionflower or musk rose oil; or even triglycerides of caprylic/capric acids, such as those sold by Stéarineries Dubois or those sold under the names Miglyol 810, 812 and 818 by Dynamit Nobel,
synthetic ethers having from 10 to 40 carbon atoms;
linear or branched hydrocarbons of mineral or synthetic origin, such as liquid petrolatum, polydecenes, hydrogenated polyisobutene, such as Parleam oil, squalane and their mixtures;
synthetic esters, such as the oils of formula R₁COOR₂ in which R₁ represents the residue of a linear or branched fatty acid comprising from 1 to 40 carbon atoms and R₂ represents a hydrocarbon chain, in particular a branched hydrocarbon chain, comprising from 1 to 40 carbon atoms, provided that R₁+R₂ is ≧10, such as, for example, Purcellin oil (cetearyl octanoate), isopropyl myristate, isopropyl palmitate, C₁₂ to C₁₅ alkyl benzoate, hexyl laurate, diisopropyl adipate, isononyl isononanoate, 2-ethylhexyl palmitate, isostearyl isostearate, octanoates, decanoates or ricinoleates of alcohols or of polyalcohols, such as propylene glycol dioctanoate; hydroxylated esters, such as isostearyl lactate or diisostearyl malate; and pentaerythritol esters;
fatty alcohols comprising a branched and/or unsaturated carbon chain having from 12 to 26 carbon atoms which are liquid at ambient temperature, such as octyldodecanol, isostearyl alcohol, oleyl alcohol, 2-hexyldecanol, 2-butyloctanol or 2-undecylpentadecanol;
higher fatty acids, such as oleic acid, linoleic acid or linolenic acid;
carbonates;
acetals;
citrates;
and their mixtures.

The nonvolatile silicone oils which can be used in the compositions according to the invention can be polydimethylsiloxanes (PDMSs) which are nonvolatile, polydimethylsiloxanes comprising pendent alkyl or alkoxy groups and/or alkyl or alkoxy groups at the end of the silicone chain, groups each having from 2 to 24 carbon atoms, phenylated silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

The fluorinated oils which can be used in the invention are in particular fluorosilicone oils, fluorinated polyethers or fluorinated silicones, such as described in the document EP-A-847 752.

Film-Forming Polymer

The first and/or the second composition according to the invention can comprise, in addition to the compound or the mixture of compounds which confers, on the second composition, a dmax of greater than or equal to 5 mm, at least one film-forming polymer.

The film-forming polymer can be present in the composition according to the invention in a content of dry matter (or active materials) ranging from 0.1% to 30% by weight, with respect to the total weight of each first and second composition, preferably from 0.5% to 20% by weight and better still from 1% to 15% by weight.

In the present invention, the term “film-forming polymer” is understood to mean a polymer capable of forming, alone or in the presence of an additional agent which is able to form a film, a macroscopically continuous film which adheres to keratinous fibres.

Mention may be made, among the film-forming polymers which can be used in the first and/or the second composition of the present invention, of synthetic polymers of radical type or of polycondensate type, polymers of natural origin, and their mixtures.

The term “radical film-forming polymer” is understood to mean a polymer obtained by polymerization of monomers possessing unsaturation, in particular ethylenic unsaturation, each monomer being capable of homopolymerizing (unlike polycondensates).

The film-forming polymers of radical type can in particular be vinyl polymers or copolymers, in particular acrylic polymers.

The film-forming vinyl polymers can result from the polymerization of monomers possessing ethylenic unsaturation having at least one acid group and/or of the esters of these acidic monomers and/or of the amides of these acidic monomers.

Use may be made, as monomer carrying an acid group, of unsaturated α,β-ethylenic carboxylic acids, such as acrylic acid, methacrylic acid, crotonic acid, maleic acid or itaconic acid. Use is preferably made of (meth)acrylic acid and crotonic acid and more preferentially of (meth)acrylic acid.

The esters of acidic monomers are advantageously chosen from esters of (meth)acrylic acid (also known as (meth)acrylates), in particular alkyl (meth)acrylates, especially C₁-C₃₀ alkyl(meth)acrylates, preferably C₁-C₂₀ alkyl(meth)acrylates, aryl(meth)acrylates, in particular C₆-C₁₀ aryl(meth)acrylates, hydroxyalkyl(meth)acrylates, in particular C₂-C₆ hydroxyalkyl(meth)acrylates.

Mention may be made, among alkyl(meth)acrylates, of methyl methacrylate, ethyl methacrylate, butyl methacrylate, isobutyl methacrylate, 2-ethylhexyl methacrylate, lauryl methacrylate or cyclohexyl methacrylate.

Mention may be made, among hydroxyalkyl(meth)acrylates, of hydroxyethyl acrylate, 2-hydroxypropyl acrylate, hydroxyethyl methacrylate or 2-hydroxypropyl methacrylate.

Mention may be made, among aryl(meth)acrylates, of benzyl acrylate and phenyl acrylate.

Esters of (meth)acrylic acid which are particularly preferred are alkyl(meth)acrylates.

According to the present invention, the alkyl group of the esters can be either fluorinated or perfluorinated, that is to say that a portion or all of the hydrogen atoms of the alkyl group are substituted by fluorine atoms.

Mention may be made, as amides of the acidic monomers, for example, of (meth)acrylamides, in particular N-alkyl(meth)acrylamides, especially N-(C₂-C₂₂ alkyl)(meth)acrylamides. Mention may be made, among N-alkyl(meth)acrylamides, of N-ethylacrylamide, N-(t-butyl)-acrylamide, N-(t-octyl)acrylamide and N-undecylacrylamide.

The film-forming vinyl polymers can also result from the homopolymerization or from the copolymerization of monomers chosen from vinyl esters and styrene monomers. In particular, these monomers can be polymerized with acidic monomers and/or their esters and/or their amides, such as those mentioned above.

Mention may be made, as examples of vinyl esters, of vinyl acetate, vinyl neodecanoate, vinyl pivalate, vinyl benzoate and vinyl t-butylbenzoate.

Mention may be made, as styrene monomers, of styrene and α-methylstyrene.

Mention may be made, among film-forming polycondensates, of polyurethanes, polyesters, polyesteramides, polyamides, epoxy ester resins or polyureas.

The polyurethanes can be chosen from anionic, cationic, nonionic or amphoteric polyurethanes, polyurethane-acrylics, polyurethane-polyvinylpyrrolidones, polyester-polyurethanes, polyether-polyurethanes, polyureas, polyurea-polyurethanes, and their blends.

The polyesters can be obtained in a known way by polycondensation of dicarboxylic acids with polyols, in particular diols, such as described above for the sulphopolyester.

The optionally modified polymers of natural origin can be chosen from shellac resin, gum sandarac, dammars, elemis, copals, cellulose polymers and their blends.

According to a first embodiment of the composition according to the invention, the film-forming polymer can be a water-soluble polymer and can be present in an aqueous phase of the composition; the polymer is thus dissolved in the aqueous phase of the composition.

According to another alternative embodiment of the invention, the film-forming polymer can be a polymer dissolved in a liquid fatty phase comprising oils or organic solvents, such as those described above (the film-forming polymer is then described as a fat-soluble polymer). Preferably, the liquid fatty phase comprises a volatile oil, optionally as a mixture with a non-volatile oil, it being possible for the oils to be chosen from the oils mentioned above.

Mention may be made, as examples of fat-soluble polymer, of copolymers of vinyl ester (the vinyl group being directly connected to the oxygen atom of the ester group and the vinyl ester having a saturated, linear or branched, hydrocarbon radical of 1 to 19 carbon atoms bonded to the carbonyl of the ester group) and of at least one other monomer which can be a vinyl ester (other than the vinyl ester already present), an α-olefin (having from 8 to 28 carbon atoms), an alkyl vinyl ether (the alkyl group of which comprises from 2 to 18 carbon atoms) or an allyl or methallyl ester (having a saturated, linear or branched, hydrocarbon radical of 1 to 19 carbon atoms bonded to the carbonyl of the ester group).

These copolymers can be crosslinked using crosslinking agents which can be either of the vinyl type or of the allyl or methallyl type, such as tetraallyloxyethane, divinylbenzene, divinyl octanedioate, divinyl dodecanedioate and divinyl octadecanedioate.

Mention may be made, as examples of these copolymers, of the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% of divinylbenzene, vinyl dimethylpropionate/vinyl laurate, crosslinked with 0.2% of divinylbenzene, vinyl acetate/octadecyl vinyl ether, crosslinked with 0.2% of tetraallyloxyethane, vinyl acetate/allyl stearate, crosslinked with 0.2% of divinylbenzene, vinyl acetate/1-octadecene, crosslinked with 0.2% of divinylbenzene, and allyl propionate/allyl stearate, crosslinked with 0.2% of divinylbenzene.

Mention may also be made, as fat-soluble film-forming polymers, of fat-soluble copolymers and in particular those resulting from the copolymerization of vinyl esters having from 9 to 22 carbon atoms or of alkyl acrylates or methacrylates, the alkyl radicals having from 10 to 20 carbon atoms.

Such fat-soluble copolymers can be chosen from copolymers of poly(vinyl stearate), of poly(vinyl stearate) crosslinked using divinylbenzene, diallyl ether or diallyl phthalate, copolymers of poly(stearyl(meth)acrylate), of poly(vinyl laurate), of poly(lauryl(meth)acrylate), it being possible for these poly(meth)acrylates to be crosslinked using ethylene glycol dimethacrylate or tetraethylene glycol dimethacrylate.

The fat-soluble copolymers defined above are known and are described in particular in Application FR-A-2 232 303; they can have a weight-average molecular weight ranging from 2000 to 500 000 and preferably from 4000 to 200 000.

Mention may also be made, as fat-soluble film-forming polymers which can be used in the invention, of polyalkylenes and in particular copolymers of C₂-C₂₀ alkenes, such as polybutene, alkylcelluloses with a saturated or unsaturated and linear or branched C₁ to C₈ alkyl radical, such as ethylcellulose and propylcellulose, copolymers of vinylpyrrolidone (VP) and in particular copolymers of vinylpyrrolidone and of C₂ to C₄₀ alkene and better still C₃ to C₂₀ alkene. Mention may be made, as examples of VP copolymer which can be used in the invention, of the VP/vinyl acetate, VP/ethyl methacrylate, VP/ethyl methacrylate/methacrylic acid, VP/eicosene, VP/hexadecene, VP/triacontene, VP/styrene or VP/acrylic acid/lauryl methacrylate copolymer or butylated polyvinylpyrrolidone (PVP).

Mention may also be made of silicone resins, generally soluble or swellable in silicone oils, which are crosslinked polyorganosiloxane polymers. The nomenclature of silicone resins is known under the name of “MDTQ”, the resin being described according to the various siloxane monomer units which it comprises, each of the letters “MDTQ” characterizing one type of unit.

Mention may be made, as examples of commercially available polymethylsilsesquioxane resins, of those which are sold:
by Wacker under the reference Resin MK, such as Belsil PMS MK;
by Shin-Etsu under the reference KR-220L.

Mention may be made, as siloxysilicate resins, of trimethylsiloxysilicate (TMS) resins, such as those sold under the reference SR1000 by General Electric or under the reference TMS 803 by Wacker. Mention may also be made of trimethylsiloxysilicate resins sold in a solvent, such as cyclomethicone, sold under the names “KF-7312J” by Shin-Etsu or “DC 749” or “DC 593” by Dow Corning.

Mention may also be made of copolymers of silicone resins, such as those mentioned above with polydimethylsiloxanes, such as the pressure-sensitive adhesive copolymers sold by Dow Corning under the reference BIO-PSA and described in the document U.S. Pat. No. 5,162,410 or the silicone copolymers resulting from the reaction of a silicone resin, such as those described above, and of a diorganosiloxane, such as are described in the document WO 2004/073626.

The film-forming polymer can also be present in the compositions in the form of particles in dispersion in an aqueous phase or in a nonaqueous solvent phase, generally known under the name of latex or pseudolatex. The techniques for the preparation of these dispersions are well known to a person skilled in the art.

Use may be made, as aqueous film-forming polymer dispersion, of acrylic dispersions, sold under the names Neocryl XK-90®, Neocryl A-1070®, Neocryl A-1090®, Neocryl BT-62®, Neocryl A-1079® and Neocryl A-523® by Avecia-Neoresins, Dow Latex 432® by Dow Chemical, Daitosol 5000 ADO or Daitosol 5000 SJ® by Daito Kasey Kogyo; Syntran 5760® by Interpolymer, Allianz OPT by Rohm & Haas, aqueous dispersions of acrylic or styrene/acrylic polymers, sold under the trade name Joncryl® by Johnson Polymer, or aqueous dispersions of polyurethane, sold under the names Neorez R-981® and Neorez R-974® by Avecia-Neoresins, Avalure UR-405®, Avalure UR-410®, Avalure UR-425®, Avalure UR-450®, Sancure 875®, Sancure 861®, Sancure 878® and Sancure 2060® by Goodrich, Impranil 85® by Bayer, Aquamere H-1511® by Hydromer; sulphopolyesters, sold under the trade name Eastman AQ® by Eastman Chemical Products, vinyl dispersions, such as Mexomer PAM® from Chimex, and their blends.

Mention may be made, as examples of nonaqueous dispersions of the film-forming polymer, of acrylic dispersions in isododecane, such as Mexomer PAP® from Chimex, dispersions of particles of a grafted ethylenic polymer, preferably an acrylic polymer, in a liquid fatty phase, the ethylenic polymer advantageously being dispersed in the absence of additional stabilizer at the surface of the particles, such as described in particular in the document WO 04/055081.

The first and/or the second composition according to the invention can comprise a plasticizing agent which promotes the formation of a film with the film-forming polymer. Such a plasticizing agent can be chosen from all the compounds known to a person skilled in the art as being capable of performing the desired function.

The first and/or the second composition can also comprise ingredients commonly used in cosmetics, such as lipophilic gelling agents, colouring materials, fillers, fibres and their mixtures.

Colouring Material

The first and/or the second composition according to the invention can also comprise at least one colouring material, such as pulverulent materials, fat-soluble dyes or water-soluble dyes.

The pulverulent colouring materials can be chosen from pigments and pearlescent agents.

The pigments can be white or coloured, inorganic and/or organic and coated or noncoated. Mention may be made, among inorganic pigments, of titanium dioxide, optionally treated at the surface, zirconium, zinc or cerium oxides, and also iron or chromium oxides, manganese violet, ultramarine blue, chromium hydrate and ferric blue. Mention may be made, among organic pigments, of carbon black, pigments of D & C type and lakes, based on cochineal carmine, of barium, strontium, calcium or aluminium.

The pearlescent agents can be chosen from white pearlescent pigments, such as mica covered with titanium oxide or with bismuth oxychloride, coloured pearlescent pigments, such as titanium oxide-coated mica with iron oxides, titanium oxide-coated mica with in particular ferric blue or chromium oxide or titanium oxide-coated mica with an organic pigment of the abovementioned type, and pearlescent pigments based on bismuth oxychloride.

The fat-soluble dyes are, for example, Sudan red, D&C Red 17, D&C Green 6, β-carotene, soybean oil, Sudan brown, D&C Yellow 11, D&C Violet 2, D&C Orange 5, quinoline yellow or annatto. These colouring materials can be present in a content ranging from 0.01 to 30% by weight, with respect to the total weight of each composition comprising them.

The first and/or the second composition used in the present invention can comprise substances exhibiting a nonzero magnetic susceptibility, also known as “magnetic substances”, which can be provided in various forms.

According to one embodiment, the second composition comprises magnetic substances.

Thus, according to one embodiment, the method according to the invention comprises the following stages:
a) application to the keratinous fibres of a first composition as defined above,
b) application to the keratinous fibres, preferably in the absence of a magnetic field, of a second cosmetic composition comprising magnetic substances and at least one compound or a mixture of compounds which, when the composition is brought to a temperature of greater than or equal to 40° C., confers, on the said composition, a dmax threading nature of greater than or equal to 5 mm,
c) bringing the said second composition, prior to, simultaneously with or subsequent to its application, to a temperature of greater than or equal to 40° C.,
d) at least partially subjecting the said second composition to a magnetic field, so as to shift and/or modify the orientation of at least some of the substances possessing nonzero magnetic susceptibility,

the order of stages b) and c) being unimportant.

The application of the composition in the absence of a magnetic field assumes the use of an applicator which is nonmagnetic or weakly magnetic at the time of application and which does not interact with the composition to the extent of significantly modifying the way in which the latter is applied. The applicator can thus advantageously be nonmagnetic and entirely conventional.

The magnetic field is, for example, generated by a magnetic device separate from the applicator or integral with the latter but sufficiently distant from the application member not to interact significantly with the composition during application.

Such a method makes it possible to obtain to reinforce the elongating effect on the eyelashes and/or a curling effect on the eyelashes and/or to improve the separation of the eyelashes.

The magnetic field can be generated so as to subject the composition present on the eyelashes to field lines which are substantially parallel to the eyelashes, for the purpose of lengthening and/or separating the eyelashes. The magnetic field can, for example, be generated by a magnet or electromagnet having a polar axis substantially in the axis of the eyelashes. Such a magnetic field promotes the shifting of the composition in the direction of the elongation of the eyelash.

The expression “magnetic substances” should not be understood in a limiting way and covers particles, fibres or agglomerates of particles and/or of fibres, of any shape, exhibiting a nonzero magnetic susceptibility.

The concentration of magnetic substances in the composition is, for example, between approximately 0.05% and approximately 50% by weight, in particular between approximately between 0.1% and approximately 40% by weight, better still between approximately 1% and approximately 30% by weight.

The composition applied can comprise magnetic fibres or other aspherical substances, such as chains of particles or of fibres.

Preferably, the magnetic substances do not exhibit residual magnetization in the absence of a magnetic field.

The magnetic substances can comprise any magnetic material exhibiting sensitivity to the lines of a magnetic field, whether this field is produced by a permanent magnet or results from induction, this material being, for example, chosen from nickel, cobalt, iron, their alloys and oxides, in particular Fe₃O₄, and also gadolinium, terbium, dysprosium, erbium, their alloys and oxides. The magnetic material can be of “soft” or “hard” type. The magnetic material can in particular be soft iron, which exhibits a high susceptibility and can facilitate the production of a thickness-extending effect.

The magnetic substances may or may not exhibit a multilayer structure, comprising at least one layer of a magnetic material, such as, for example, iron, nickel, cobalt, their alloys and oxides, in particular Fe₃O₄.

The magnetic substances are preferably aspherical, for example exhibiting an elongated form. Thus, when these substances are subjected to the magnetic field, they tend to orientate themselves with their longitudinal axis aligned with the field lines and undergo a change in orientation which is reflected by a change in appearance of the composition.

When the magnetic substances are substantially spherical particles, their appearance is preferably nonhomogeneous, so that a change in orientation brings about a change in appearance.

The size of the substances, whatever their form, is, for example, between 1 nm and 10 mm, better still between 10 nm and 5 mm, even better still between 100 nm and 1 mm, for example between 0.5 μm and 300 μm or 1 μm and 150 μm. The size is that given by the statistical distribution with respect to half the population, referred to as D50.

When the substances are particles not having an elongated form or having an elongated form with a fairly low aspect ratio, the size of the particles is, for example, less than 1 mm.

The magnetic substances are, for example, magnetic pigments, magnetic composite particles, magnetic fibres, ferrofluids or chains of magnetic particles and/or fibres.

Magnetic Pigments

Pigments which are very particularly suitable are pearlescent agents comprising iron oxide Fe₃O₄. Pigments exhibiting magnetic properties are, for example, those sold under the trade names Colorona Blackstar Blue, Colorona Blackstar Green, Colorona Blackstar Gold, Colorona Blackstar Red, Cloisonné Nu Antique Super Green, Microna Matte Black (17437), Mica Black (17260), Colorona Patina Silver (17289) and Colorona Patina Gold (117288) from Merck or alternatively

Flamenco Twilight Red, Flamenco Twilight Green, Flamenco Twilight Gold, Flamenco Twilight Blue, Timica Nu Antique Silver 110 AB, Timica Nu Antique Gold 212 GB, Timica Nu Antique Copper 340 AB, Timica Nu Antique Bronze 240 AB, Cloisonné Nu Antique Green 828 CB, Cloisonné Nu Antique Blue 626 CB, Gemtone Moonstone G 004, Cloisonné Nu Antique Red 424 CB, Chroma-Lite Black (4498), Cloisonné Nu Antique Rouge Flambé (code 440 XB), Cloisonné Nu Antique Bronze (240 XB), Cloisonné Nu Antique Gold (222 CB) and Cloisonné Nu Antique Copper (340 XB) from Engelhard.

Mention may be made, as further example of magnetic pigment capable of participating in the formulation of the composition, of particles of black iron oxide, for example those sold under the name Sicovit Black E172 by BASF.

The magnetic pigments can also comprise iron metal, in particular passivated soft iron, for example obtained from iron carbonyl by employing the process described in Patent U.S. Pat. No. 6,589,331. These particles can comprise a surface layer of an oxide.

Magnetic Fibres

The term “fibres” denotes generally elongated substances, for example exhibiting an aspect ratio ranging from 3.5 to 2500 or from 5 to 500, for example from 5 to 150. The aspect ratio is defined by the L/D ratio, where L is the length of the fibre and D is the diameter of the circle in which the greatest transverse cross section of the fibre is framed. The transverse cross section of the fibres can be framed, for example, in a circle with a diameter ranging from 2 nm to 500 μm, for example ranging from 100 nm to 100 μm, indeed even from 1 μm to 50 μm.

The fibres can, for example, exhibit a length ranging from 1 μm to 10 mm, for example from 0.1 mm to 5 mm, indeed even from 0.3 mm to 3.5 mm.

The fibres can exhibit a weight ranging, for example, from 0.15 to 30 denier (weight in grams per 9 km of yarn), for example from 0.18 to 18 denier.

The fibres can have any shape in transverse cross section, for example circular or polygonal, in particular square, hexagonal or octogonal.

The composition can comprise solid or hollow fibres which are independent or bonded to one another, for example plaited.

The composition can comprise fibres having ends which are blunted and/or rounded, for example by polishing.

The fibres may not experience a substantial modification to their form when they are introduced in the composition, for example being initially rectilinear and sufficiently rigid to retain their form. In a variant, the fibres may exhibit a flexibility which allows them to substantially change shape within the composition.

The fibres can comprise a nonzero content, which can range up to 100%, of a magnetic material chosen from soft magnetic materials, hard magnetic materials, in particular based on iron, on zinc, on nickel, on cobalt or on manganese and their alloys and oxides, in particular Fe₃O₄, rare earth metals, barium sulphate, iron-silicon alloys, optionally charged with molybdenum, Cu₂MnAl, MnBi, or a mixture of these, this list not being limiting.

When the composition comprises fibres comprising magnetic particles, the latter can be present, for example, at least at the surface of the fibre, indeed even at the surface of the fibres only, or inside the fibre only, or also can be dispersed within the fibre in a substantially homogeneous fashion.

The fibres can comprise, for example, a nonmagnetic core with a plurality of magnetic particles at its surface.

The fibres can also comprise a synthetic matrix comprising a plurality of magnetic grains dispersed within it.

If appropriate, a synthetic material charged with magnetic particles can itself be coated with a nonmagnetic shell. Such a shell constitutes, for example, a barrier which isolates the magnetic material or materials from the ambient medium and/or can introduce colour. The fibres can comprise a monolithic magnetic core and be coated with a nonmagnetic shell, or this situation can be inverted.

The composition can comprise fibres produced by extrusion or coextrusion of one or more polymers, in particular thermoplastics and/or elastomers. One of the materials extruded can comprise a charge of dispersed magnetic particles.

The fibre can comprise a synthetic material chosen from polyamides, PET, acetates, polyolefins, in particular PE or PP, PVC, polyester-block-amide, plasticized Rilsan®, elastomers, in particular polyester elastomers, PE elastomers, silicone elastomers, nitrile elastomers or a blend of these materials, this list not being limiting.

The composition can comprise composite fibres comprising a magnetic core at least partially coated with at least one synthetic or natural nonmagnetic material. The coating of the magnetic core can be carried out, for example, by coextrusion, around the core, of a shell made of a nonmagnetic material.

The coating of the core can also be carried out in another way, for example by in situ polymerization.

The core can be monolithic or can comprise a charge of magnetic grains dispersed in a matrix.

The composition can also comprise composite fibres obtained by coating a synthetic or natural nonmagnetic core with a synthetic material charged with magnetic particles, the core being composed, for example, of a fibre made of wood, rayon, polyamide, a plant material, polyolefin, in particular polyethylene, Nylon®, polyimideamide or aramid, this list not being limiting.

Magnetic particles and applicational devices are described in particular in the document WO 06/037900.

Fillers

The second composition according to the invention can additionally comprise at least one filler as defined above.

The compositions of the invention can additionally comprise any additive conventionally used in cosmetics, such as antioxidants, preservatives, fibres, fragrances, neutralizing agents, gelling agents, thickeners, vitamins, coalescence agents, plasticizers and their mixtures.

Fibres

The first and/or the second composition according to the invention can comprise, in addition to the compound or the mixture of compounds which confers, on the said composition, a dmax threading nature of greater than or equal to 5 mm, “additional” fibres. These additional fibres alone do not contribute to the threading nature of the composition.

The additional fibres can be present in the composition according to the invention in a content ranging from 0.01% to 10% by weight, with respect to the total weight of the composition comprising them, in particular from 0.1% to 5% by weight and more particularly from 0.3% to 3% by weight.

Cosmetic Active Principles

Mention may in particular be made, as cosmetic active principles which can be used in the compositions according to the invention, of antioxidants, preservatives, fragrances, neutralizing agents, emollients, moisturizing agents, vitamins and screening agents, in particular sunscreens.

Of course, a person skilled in the art will take care to choose the optional additional additives and/or their amounts so that the advantageous properties of the compositions according to the invention are not, or not substantially, detrimentally affected by the envisaged addition.

The first and second compositions can be provided in the solid, semisolid or liquid form.

The first and second compositions can be provided in particular in the form of a suspension, dispersion, solution, gel, emulsion, in particular oil-in-water (O/W), wax-in-water or water-in-oil (W/O), or multiple (W/O/W or polyol/O/W or O/W/O) emulsion, cream, foam, dispersion of vesicles, in particular of ionic or nonionic lipids, two-phase or multiphase lotion, spray, powder or paste, in particular soft paste. Each composition is preferably a leave-in composition.

The first and second compositions according to the invention can be manufactured by known processes generally used in the cosmetics field.

The first composition can be applied to the eyelashes using a conventional mascara applicator, in particular in the form of a brush comprising an arrangement of hairs held by a twisted wire. Such a twisted brush is described in particular in U.S. Pat. No. 4,887,622. It can also be in the form of a comb comprising a plurality of applicational parts, obtained in particular from moulding. Such combs are described, for example, in Patent FR 2 796 529. The applicator can be integral with the container, such as described, for example, in Patent FR 2 761 959. Advantageously, the applicator is integral with a rod which, itself, is integral with the closure part. In the context of the method according to the invention, the first composition is in particular applied to the keratinous fibres using a mascara brush.

The first composition is provided in particular in the form of a wax-in-water emulsion.

In the method of the invention, the second composition is generally heated to a temperature of greater than or equal to 40° C., in particular of greater than or equal to 45° C., especially of greater than or equal to 50° C.

Obviously, the heating temperature depends in particular on the temperature capable of being withstood by the treated support.

According to one embodiment, the second composition is in the solid form.

According to one embodiment, the first composition is provided in the form of a wax-in-water emulsion and the second composition is in the solid form.

According to a first embodiment of the method according to the invention, the second composition is solid and is heated prior to the application thereof, it being possible for the heating means used to be the applicator itself. Thus, in the case of a mascara, the second composition can be applied using a heating applicator, such as a heating brush.

According to another embodiment of the method according to the invention, the second composition is heated during the application thereof. In such a case, the heating means used is generally the applicator itself. Thus, in the case of a mascara, the second composition can be applied using a heating brush.

According to a second embodiment, the second composition is heated subsequent to the application thereof. According to a first alternative form, the second composition can be heated using means not specifically intended for a heating operation, such as, for example, a body which is occasionally warm. According to a second alternative form of this embodiment, the composition can be heated using a means specifically dedicated to the heating. It can in particular be a means which propels hot air, such as a hairdryer, or a heating device, as described below.

According to one embodiment, the second composition according to the invention is in the form of particles, of a powder or of a pulverulent mass. This second composition can be applied to the keratinous fibres using an applicational device comprising a heating support, the composition being present in an applicator nozzle having a shape suited to the fitting thereof by coupling to the heating support or the composition being present in a container in which the heating support can be immersed in order to become charged with composition.

According to one embodiment, the second composition, which is provided in a powder form, is placed on the heating part of a heating applicator, such as a heating brush or comb, until it softens and then it is applied to the keratinous fibres.

The second composition according to the invention can be packaged in a packaging and applicational combination comprising:
i) a reservoir comprising the said composition,
ii) a device for the application of the composition, and
iii) heating means.

According to one embodiment, the heating means are formed by a device separate from the applicational device or member, the combination being configured in the form of a packaging and applicational device additionally comprising a container comprising a second composition in accordance with the invention. Such a device can be packaged inside a packaging of the blister pack type. The heating means can be of the type of those described in U.S. Pat. No. 6,009,884 or U.S. Pat. No. 5,853,010. Other devices configured in the form of heating tongs (in the case of the eyelashes) can also be used. Such devices are described in particular in U.S. Pat. No. 6,220,252.

According to one embodiment, the applicational device or member comprises means for heating the second composition; in particular, the heating means associated with the applicational device are arranged so as not to substantially heat at least a portion of the rod.

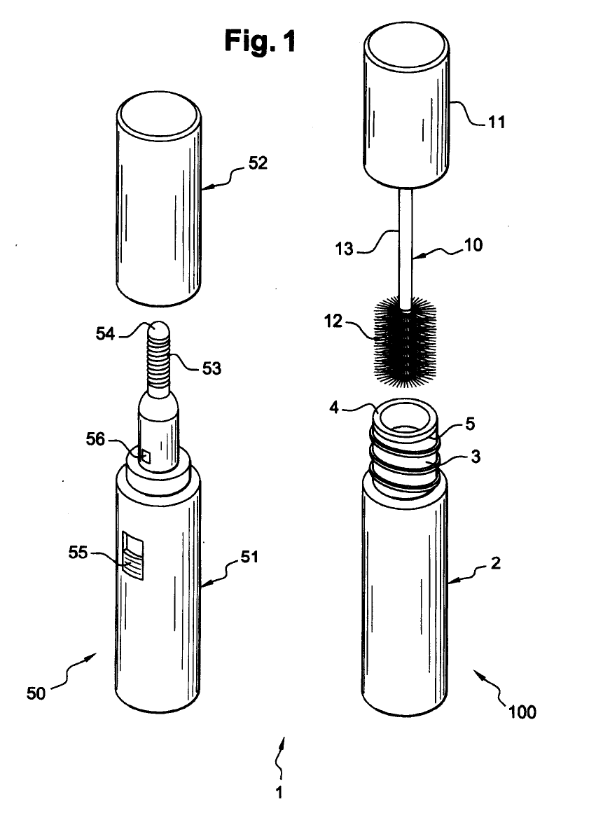

The kit 1 described in FIG. 1 comprises a mascara packaging and applicational combination 100 and a heating device 50 which is separate from the packaging and applicational combination.

The two devices 100 and 50 can be sold together in the same packaging of blister pack type. The unit 100 comprising the product can be sold separately.

The packaging and applicational combination 100 comprises a container 2, comprising the second composition according to the invention, on which is mounted a threaded neck 3, a free edge of which delimits an opening 4. A draining member 5 is mounted in the opening 4. The combination 100 also comprises an applicational device 10 comprising a stopper 11 integral with a rod 13, one end of which comprises an applicator 12 generally configured in the form of an arrangement of fibres held between the two branches of a twisted iron wire. An internal surface of the stopper 11 is threaded so as to engage with the threading of the neck 3. Thus, when the applicator 12 and the rod 13 are positioned inside the container 2, the threading of the stopper 11 engages with the threading of the neck 3 so that the stopper seals in a leaktight fashion the opening 4 of the container. Such packaging and applicational combinations are well known.

The heating device 50 is in accordance with what is described in U.S. Pat. No. 6,009,884. It mainly comprises a grasping portion 51 and a cap 52. A battery is positioned inside the grasping portion 51 and is connected to a heating wire 53 configured in the form of a helical winding positioned on a rod 54. A switch 55 makes it possible to apply a voltage to the device and respectively to turn off the voltage. An LED 56, when it changes colour, indicates that the device is at the required temperature and that it is thus ready to be used.

The supply to the heating portion via the battery is 12 V. The power dissipated is approximately 1 watt. The heating wire 53 can be made of a nickel/chromium alloy.

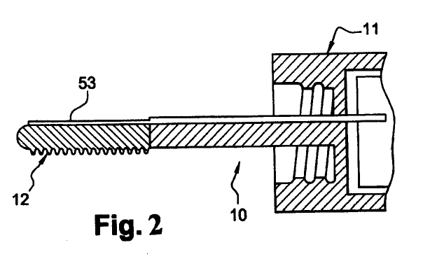

In the embodiment of FIG. 2, the applicator 12 is composed of a metal cylinder, at least a portion of the periphery of which is fluted perpendicular to its longitudinal axis. The fluted cylinder is attached, in particular by adhesive bonding, to the end of the rod 13. A heating resistance 53, which extends over substantially the entire length of the applicator 12, is positioned diametrically opposite the fluted portion. The heating resistance 53 can be positioned in a groove made longitudinally in the surface of the cylinder.

Thus, the heating resistance 53 heats the second composition present on the fluted cylinder, the fluted region of the latter being used for the application proper of the product on the eyelashes and for their separation.

According to one embodiment, the second composition is applied to the eyelashes conventionally under cold conditions using a brush 12 and is then heated after application: the user engages the heating portion 53 of the device 50 with the eyelashes so as to bring the deposited layer of product to the threading temperature of the second composition and then drawn out by means of the heating device formed on the eyelashes so as to create threads in the extension of the eyelashes.

On cooling, the threads are set in the extension of the eyelashes, making it possible to obtain an elongating effect.

According to another embodiment, the mascara is in a solid form and is used with a heating device 50 alone. It is brought into contact with the heating portion 53 of the device 50 and then heated so as to bring the deposited layer of product to the threading temperature of the second composition. The user then engages the heating portion 53 of the device with the eyelashes and then draws out, by means of the device, the deposited layer formed on the eyelashes so as to create threads in the extension of the eyelashes.

The examples which appear below are presented by way of illustration and without implied limitation of the invention. Unless otherwise indicated, the amounts shown are expressed as percentage by weight.

### EXAMPLE 1

### Mascara Kit

Mascaras were prepared which have the following composition:

First Compositions

| | 1A | 1B | 1C | 1D |
|---|---|---|---|---|
| trans-Isomerized | 4.04 | 4.04 | 4.04 | 4.04 |
| jojoba oil with | | | | |
| M.p. = 45° C. (Iso- | | | | |
| Jojoba-50 ® from Desert | | | | |
| Whale) | | | | |
| Paraffin wax (Cerafine | 12.77 | 12.77 | — | 12.77 |
| 56/58 from Baerlocher) | | | | |
| Carnauba wax | 3.19 | 3.19 | 3.19 | 15.96 |
| Tetrafluoroethylene | — | — | 12.77 | — |
| powder (M.p. > 600° C.) | | | | |
| (Microslip 519 L ® from | | | | |
| Micro Powders) | | | | |
| 2-Octyldodecyl | — | 5 | — | — |
| neopentanoate | | | | |
| Black iron oxide | 7.14 | 7.14 | 7.14 | 7.14 |
| Gum arabic | 3.39 | 3.39 | 3.39 | 3.39 |
| Hydroxyethylcellulose | 0.89 | 0.89 | 0.89 | 0.89 |
| Potassium cetyl | 2.53 | 2.53 | 2.53 | 2.53 |
| phosphate (Amphisol K | | | | |
| from Givaudan) | | | | |
| Oxyethylenated stearyl | 5.16 | 5.16 | 5.16 | 5.16 |
| alcohol (20 mol of OE) | | | | |
| (Brij 78 from Uniqema) | | | | |
| Oxyethylenated stearyl | 2.44 | 2.44 | 2.44 | 2.44 |
| alcohol (2 mol of OE) | | | | |
| (Brij 72 from Uniqema) | | | | |
| Cetyl alcohol | 2.32 | 2.32 | 2.32 | 2.32 |
| Simethicone | 0.13 | 0.13 | 0.13 | 0.13 |
| Ethanol | 3 | 3 | 3 | 3 |
| Preservatives | q.s. | q.s. | q.s. | q.s. |
| Water | q.s. | q.s. | q.s. | q.s. |
| | for 100 | for 100 | for 100 | for 100 |

Second Composition

| Phase A | |
|---|---|
| Mixture of paraffin wax (30-50%) and of ethylene/vinyl | 95% |
| acetate copolymer comprising 28% of vinyl acetate (50 to | |
| 65%) | |
| Phase B | |
| Black iron oxide | 5% |

Procedure:

This composition is prepared in a twin-screw mixer/extruder (Prism type from Thermo Electron Corporation, United Kingdom) comprising 6 independent barrels each making it possible to introduce a fresh phase and to set the temperature. They are numbered from 1 to 6 from the inlet to the outlet for the product. The flow rate is 2 kg/h (1000 rpm).

The ingredients of phases A and B are introduced respectively into the first and second barrels, in which they are heated to 100° C., and then the 2 phases are mixed under hot conditions.

The composition is subsequently cooled in an ice chest in order to obtain small slabs of solid formulation, which are subsequently subjected to cryogenic milling on a Quadro mill at −200° C. on a 0.5 mm sieve. A composition in the form of a pulverulent mass is obtained.

Each of the compositions 1A to 1D is applied to a test specimen of eyelashes (referenced A, B, C and D) using a conventional mascara brush.

The second composition in the powder form is subsequently placed on the heating portion of a heating applicator (of heating brush type), the composition softens and then the second composition is applied to the end of the eyelashes, coated with the first composition, of each test specimen while drawing out the deposited layer using the applicator.

This second composition is also applied to the end of the eyelashes of a test specimen of “bare” eyelashes not coated with the first composition (known as test specimen E).

The second compositions exhibits a hot threading nature at approximately 70° C. with threads with a mean length of 35 mm. The threads obtained are fine, rigid and black. They are sufficiently rigid not to bend under their own weight, remaining vertical.

The ease of removing make-up from the test specimens is subsequently evaluated according to the following protocol: each test specimen is pinched for 10 seconds in a cotton wool swab impregnated with Effacil make-up-removing lotion from Lancôme and then the cotton wool swab is drawn off in order to remove make-up from the test specimen.

The following results are obtained:

| | A | B | C | D | E |
|---|---|---|---|---|---|
| | (1^{st} composition | (1^{st} composition | (1^{st} composition | (1^{st} composition | (2^{nd} |
| Test | 1A + 2^{nd} | 1B + 2^{nd} | 1C + 2^{nd} | 1D + 2^{nd} | composition |
| specimen | composition) | composition) | composition) | composition) | alone) |
| Ease of | Moderate | Easy | Easy | Easy | Difficult |
| removing | | | | | |
| make-up | | | | | |

The application to the eyelashes of a first base coat composition as described above, before the second composition, makes it possible to facilitate the removal of the mascara film.

Use may be made, as second composition, of the mascara compositions of the following Examples 2, 3, 4, 5, 6, 7 and 8:

### EXAMPLE 2

A mascara was prepared which has the following composition:

| | |
|---|---|
| Coolbind 34-1300 from National Starch | 20% |
| Octyldodecanol | 8.67% |
| 79.5% Potassium cetyl phosphate as a mixture | 3.57% |
| with phosphoric acid, cetyl alcohol and water | |
| (Arlatone MAP 160K from Givaudan) | |
| Peg-30 glyceryl stearate | 8.67% |
| Acrylamide/AMPS Na copolymer in isohexadecane | 0.96% AM |
| with polysorbate 80 (Simulgel 600 ® from SEPPIC) | |
| Preservatives | q.s. |
| Black iron oxide | 2.4% |
| Water | q.s. for 100% |

Procedure:

The ethylene/vinyl acetate, the octyldodecanol, the PEG-30 glyceryl stearate, the potassium cetyl phosphate and the pigments are mixed vigorously under hot conditions (approximately 95° C.)

The emulsion is subsequently prepared by adding a portion of the aqueous phase (water and acrylamide/sodium acryloyldimethyltaurate copolymer and preservatives) heated to 85° C. and then, after stirring vigorously at 80° C. for 10 minutes, the remainder of the aqueous phase, which has remained at ambient temperature, is added.

The mascara obtained is black, glossy and smooth. It exhibits a hot threading nature with threads with a mean length of 20 mm. The threads obtained are fine, flexible and black.

### EXAMPLE 3

### Mascara

1/ Preparation of a poly(isobornyl acrylate/isobutyl methacrylate/isobutyl acrylate) Polymer

100 g of isododecane are introduced into a 1 litre reactor and then the temperature is increased so as to change from ambient temperature (25° C.) to 90° C. in 1 hour.

120 g of isobornyl acrylate, 90 g of isobutyl methacrylate, 110 g of isododecane and 1.8 g of 2,5-bis(2-ethylhexanoylperoxy)-2,5-dimethylhexane (Trigonox® 141 from Akzo Nobel) are subsequently added at 90° C. over 1 hour.

The mixture is maintained at 90° C. for 1 h 30. 90 g of isobutyl acrylate, 90 g of isododecane and 1.2 g of 2,5-bis(2-ethylhexanoylperoxy)-2,5-dimethylhexane are subsequently introduced into the preceding mixture, still at 90° C. and in 30 minutes.

The mixture is maintained at 90° C. for 3 hours and is then cooled.

A solution comprising 50% of polymer active material in isododecane is obtained.

A polymer is obtained comprising a first poly(isobornyl acrylate/isobutyl methacrylate) block having a Tg of 75° C., a second polyisobutyl acrylate block having a Tg of −20° C. and an intermediate block which is a random isobornyl acrylate/isobutyl methacrylate/isobutyl acrylate polymer is obtained.

This polymer exhibits a weight-average molecular weight of 144 200 g/mol and a number-average molecular weight of 49 300, i.e. a polydispersity index I of 2.93.

2/ The Following Mascara is Prepared:

| | |
|---|---|
| Poly(isobornyl acrylate/isobutyl | 58.54% |
| methacrylate/isobutyl acrylate) | |
| polymer | |
| Pigments (iron oxide) | 2.44% |
| Isododecane | q.s. for 100% |

The dmax of the composition is measured according to the protocol indicated above: this mascara exhibits a dmax of approximately 12 mm.

### EXAMPLE 4

### Mascara

| | |
|---|---|
| Styrene/butadiene copolymer | 90% |
| (Pliolite S5E from Eliokem) | |
| Isododecane | 10% |

This mascara exhibits a dmax threading nature, measured according to the protocol indicated above, of approximately 24 mm.

### EXAMPLE 5

### Mascara

A solid mascara comprising 100% of polyvinyl acetate homopolymer (Raviflex BL1S from Vinavil) is prepared.

This mascara exhibits a dmax threading nature, measured according to the protocol indicated above, of approximately 29 mm.

The composition is placed on the heating portion of a heating applicator, the composition softens and then the mascara is applied to the eyelashes while drawing out the deposited layer using the applicator.

### EXAMPLE 6

### Mascara

A mascara was prepared which has the following composition:

| | |
|---|---|
| Ethylene/vinyl acetate copolymer | 41.47% |
| (Elvax 205W from DuPont) | |
| Hydrogenated styrene/methylstyrene/ | 48.53% |
| indene/styrene copolymer | |
| (Regalite R1100 from Eastman) | |
| Paraffin wax | 10% |

The ingredients are mixed at 140° C. and then the mixture is allowed to cool to ambient temperature.

A solid mascara is obtained which is white in colour.

The dmax of the composition is measured according to the protocol indicated above: this mascara exhibits a dmax of approximately 30 mm. After heating, this mascara forms fine, flexible and transparent threads.

### EXAMPLE 7

A mascara was prepared which has the following composition:

| | |
|---|---|
| Coolbind 34-1300 from National Starch | 96% |
| Black iron oxide | 4% |

The ingredients are mixed at 100° C. and then the mixture is allowed to cool to ambient temperature.

A solid mascara is obtained which is black in colour.

The dmax of the composition is measured according to the protocol indicated above: this mascara exhibits a dmax of approximately 35 mm.

### EXAMPLE 8

A mascara can be prepared which has the following composition:

| | |
|---|---|
| Polyphenylsiloxane T resin | 98% |
| (Dow Corning (R) Z-6018 intermediate) | |
| Pigments | 2% |

The ingredients are mixed at 100° C. and then the mixture is allowed to cool to ambient temperature.

A coloured solid mascara is obtained.

The dmax of the composition is measured according to the protocol indicated above: this mascara exhibits a dmax of approximately 25 mm.

After heating, this mascara forms coloured, rigid and thick threads.

1. A cosmetic method for making up or for the nontherapeutic care of keratinous fibres comprising the application, to the keratinous fibres:
of a first composition comprising in a cosmetically acceptable medium a first compound which is an a) oil or a b) compound exhibiting a melting point or a glass transition temperature of greater than 40° C. or a mixture thereof, and
of a second composition comprising at least one compound or a mixture of compounds which, when the composition is brought to a temperature of greater than or equal to 40° C., confers, on the said composition, a dmax threading nature of greater than or equal to 5 mm, the said second composition being, prior to, simultaneously with or subsequent to the application thereof, brought to a temperature of greater than or equal to 40° C.

2. The cosmetic method according to claim 1, wherein the b) compound has a melting point or a glass transition temperature of greater than 45° C.

3. The cosmetic method according to claim 1, wherein the first compound is present in a content ranging from 0.1 to 70% by weight, with respect to the total weight of the first composition.

4. The cosmetic method according to claim 1, wherein the first compound exhibiting a first-order transition temperature of greater than 40° C. comprises particles which are solid at 25° C.

5. The cosmetic method according to claim 1, wherein the compound exhibiting a first-order transition temperature of greater than 40° C. is selected from the group consisting of waxes, fillers, metal oxides, metal particles and their mixtures.

6. The cosmetic method according to claim 1, wherein the first compound comprises at least one wax having a melting point of greater than or equal to 60° C.

7. The cosmetic method according to claim 1, wherein the first composition further comprises at least one powder, comprising a tetrafluoroethylene polymer.

8. The cosmetic method according to claim 1, wherein the first compound is at least one nonvolatile oil.

9. The cosmetic method according to claim 8, wherein the at least one nonvolatile oil is choses an ester oil.

10. The cosmetic method according to claim 1, wherein the dmax is greater than or equal to 7 mm.

11. The cosmetic method according to claim 1, wherein the second composition is brought to the temperature at which it exhibits the dmax threading nature.

12. The cosmetic method according to claim 11, wherein the temperature is greater than or equal to 45° C.

13. The cosmetic method according to claim 12, wherein the temperature is up to 150° C.

14. The cosmetic method according to claim 1, wherein the compound or the mixture of compounds which confers, on the said second composition, a dmax threading nature of greater than or equal to 5 mm exhibits a thermoplastic behaviour.

15. The cosmetic method according to claim 1, wherein the compound or the mixture of compounds which confers, on the said second composition, a dmax threading nature of greater than or equal to 5 mm is selected from the group consisting of:
a) polymers and copolymers comprising at least one alkene monomer,
b) polyvinyl acetate homopolymers,
c) silicone resins,
d) film-forming block ethylenic polymers, which comprise at least one first block and at least one second block having different glass transition temperatures (Tg), the said first and second blocks being connected to one another via an intermediate block comprising at least one constituent monomer of the first block and at least one constituent monomer of the second block,
e) copolymers of dienes and of styrene,
f) sulphopolyesters,
g) waxes,
h) fibres, and their mixtures.

16. The cosmetic method according to claim 1, wherein the compound or the mixture of compounds which confers, on the said second composition, a dmax threading nature of greater than or equal to 5 mm is selected from the group consisting of:
copolymers of alkene and of vinyl acetate,
copolymers of ethylene and of octene,
polyvinyl acetate homopolymers,
T silicone resins,
film-forming block ethylenic copolymers comprising alkyl methacrylates, alkyl acrylates or their mixtures,
copolymers of butadiene and of styrene,
copolymers obtained by condensation of diethylene glycol, cyclohexanedimethanol, isophthalic acid and sulphoisophthalic acid,
and their mixtures.

17. The cosmetic method according to claim 16, wherein the copolymer of an alkene and vinyl acetate is a copolymer of ethylene and vinyl acetate.

18. The cosmetic method according to claim 16, wherein the compound or the mixture of compounds which confers, on the said second composition, a dmax threading nature of greater than or equal to 5 mm comprises a wax and a copolymer of ethylene and vinyl acetate.

19. The cosmetic method according to claim 18, wherein the wax is a paraffin wax.

20. The cosmetic method according to claim 18, wherein a content of the copolymer of ethylene and of vinyl acetate is from 50 to 65% by weight of the total weight of the mixture.

21. The cosmetic method according to claim 18, wherein a content of the paraffin wax is from 35 to 50% by weight with respect to the total weight of the mixture.

22. The cosmetic method according to claim 17, wherein a content of vinyl acetate is more than 25% by weight with respect to the total weight of the polymer.

23. The cosmetic method according to claim 17, wherein a weight-average molecular weight (Mw) of the ethylene/vinyl acetate copolymer is from 50 000 to 80 000.

24. The cosmetic method according to claim 1, wherein a dry matter content of the compound or the mixture of compounds which confers, on the said second composition, a dmax threading nature of greater than or equal to 5 mm is greater than or equal to 5% by weight, with respect to the total weight of the composition.

25. The cosmetic method according to claim 1, wherein a dry matter content of the compound or the mixture of compounds which confers, on the said second composition, a dmax threading nature of greater than or equal to 5 mm is from 5 to 100% by weight, with respect to the total weight of the composition.

26. The cosmetic method according to claim 1, wherein the first composition comprises an aqueous phase.

27. The cosmetic method according to claim 26, wherein a content of the aqueous phase is from 5% to 95% by weight, with respect to the total weight of the first composition.

28. The cosmetic method according to claim 1, wherein the first composition and the second composition are mascaras.

29. The cosmetic method according to claim 1, wherein the second composition is a solid.

30. The cosmetic method according to claim 1, wherein a water content of the second composition is less than 20% by weight, with respect to the total weight of the composition.

31. The cosmetic method according to claim 1, wherein the second composition comprises a form selected from the group consisting of particles, of a powder and of a pulverulent mass.

32. The cosmetic method according to claim 1, wherein the second composition is brought to a temperature of greater than or equal to 40° C. prior to, simultaneously with or subsequent to the application thereof to the keratinous fibres with a heating device for application.

33. The cosmetic method according to claim 1, wherein the second composition is applied over the upper end of the keratinous fibres.

34. A kit for making up and/or caring for keratinous fibres, comprising:
a first composition comprising in a cosmetically acceptable medium a first compound which is an a) oil or a b) compound exhibiting a melting point or a glass transition temperature of greater than 40° C. or a mixture thereof, and
a second composition for making up and/or caring for keratinous fibres, the said composition comprising at least one compound or a mixture of compounds which, when the composition is brought to a temperature of greater than or equal to 40° C., confers, on the said composition, a dmax threading nature of greater than or equal to 5 mm, and
a heating device, for the application of the said make-up and/or care compositions which prior to, simultaneously with or subsequent to the application thereof, heats the said second composition to a temperature of greater than or equal to 40° C.
